(19) United States
(12) Patent Application Publication
(10) Pub. No.: US 2007/0088442 A1
(43) Pub. Date: Apr. 19, 2007

(54) PASSIVE WEAR-INDICATING SENSOR FOR IMPLANTABLE PROSTHETIC DEVICE

(75) Inventors: Michael J. Cima, Winchester, MA (US); John T. Santini JR., North Chelmsford, MA (US); Kurt Shelton, Somerville, MA (US); James H. Prescott, Cambridge, MA (US); Mark A. Staples, Cambridge, MA (US); Jonathan R. Coppeta, Windham, NH (US)

Correspondence Address: SUTHERLAND ASBILL & BRENNAN LLP, 999 PEACHTREE STREET, N.E., ATLANTA, GA, 30309, US

(73) Assignee: MICROCHIPS, INC., Bedford, MA (US)

(21) Appl. No.: 11/549,831

(22) Filed: Oct. 16, 2006

Related U.S. Application Data
(60) Provisional application No. 60/726,937, filed on Oct. 14, 2005.

(51) Int. Cl. A61F 2/30; A61F 2/28; A61B 6/12

(52) U.S. Cl. 623/18.11; 623/23.51; 623/22.13; 600/431

(57) ABSTRACT

A method is provided for non-invasively detecting mechanical wear of a prosthetic device implanted in a patient, the method comprises using a non-invasive imaging technique to image the prosthetic device that includes a wear indicating composition; and detecting whether the wear indicating composition has been released from the prosthetic device, and, if so, the location, type, and/or amount thereof. The implant device includes a prosthetic device body having at least one outer surface area; at least one reservoir (e.g., a plurality of discretely spaced micro-reservoirs) in the device body; a wear indicator composition disposed in said at least one reservoir, wherein mechanical wear of the at least one outer surface area of the device body in vivo causes release of at least part of the wear indicator composition. The prosthetic device body may be one for replacement of a hip, a knee, a shoulder, an elbow, or a vertebra.

### CROSS-REFERENCE TO RELATED APPLICATION

This application claims the benefit of U.S. Provisional Application No. 60/726,937, filed Oct. 14, 2005. The application is incorporated herein by reference in its entirety.

### BACKGROUND OF THE INVENTION

This invention is generally in the field of sensors and non-invasive means for detecting mechanical wear of devices, such as medical devices for implantation into a human or animal patient, including but not limited to orthopedic and dental prosthetic implants.

In devices where two surfaces are in contact with, and moving with respect to, one another, friction and wear can damage the contacting surfaces and affect the performance of the device. In automotive engines, for example, inadequate lubrication of the metal surfaces of the piston and cylinder can cause severe, irreversible damage to those surfaces and result in poor engine performance. Similarly, particles present in the oil lubricating the piston and cylinder surfaces can scratch the contacting surfaces, ruining their smooth finish and adversely affecting performance. Poor lubrication can result in small particles being formed by the two surfaces rubbing together (abrasion).

A similar example exists in healthcare, particularly in the field of medical implants. For example, in orthopedic implants, such as artificial joints, the implant is subjected to everyday motion, stress and strain. This often leads to abrasion between different parts of the implant, and/or between the implant and the skeletal frame of the patient in whom the device is implanted. The abrasion may generate wear debris particles in the area of the implant, and this debris can lead to serious complications. In hip, knee, shoulder, and other joint prostheses, two surfaces are in contact and rubbing against one another. In a typical total hip replacement, the surface of the head of the artificial femur (the “ball” in a ball & socket joint) rubs against an acetabular cup implant (the “socket” in the ball & socket joint) positioned in the pelvis. In many cases, the ball is made of metal (e.g., pure metals and alloys of Ti, Cr, Co, Mo, Fe, and Ni), but also may be made of ceramic materials such as alumina. The inside of the socket contacting the ball is typically made of a polymer, such as ultra-high-molecular-weight polyethylene (UHMWPE), but the socket also may be made of metal or ceramic materials such as alumina. The rubbing of the surface of the ball and the surface of the socket during normal use of the artificial hip can create abrasive wear debris or tiny particles of metal, ceramic, or polymer. Similar wear problems may occur in other types of prosthetic implants, such as knee or spinal disc replacement. The processing (e.g., sterilization method, forming method, degree of cross-linking, etc.) of some materials, such as UHMWPE, can affect the wear properties and debris generating potential of the materials as well. This debris is problematic. In particular, over time the debris can cause osteolysis, or local degradation of bone. Osteolysis is a devastating problem because local bone erosion can quickly weaken the bone remaining after the implantation of an orthopedic prosthesis, causing implant loosening, or a sudden bone or implant fracture.

Accordingly, orthopedic and spinal companies have expressed much interest in being able to detect wear and debris formation, particularly at an early enough stage to allow physicians to intervene, such as with pharmaceuticals or otherwise, before significant bone deterioration or other complications occur. For example, if the wear of one or more of the articulating surfaces could be monitored, physicians could identify those patients with implants that are generating excessive particles and will be most likely to suffer from osteolysis. The physician could then closely monitor those patients for signs of local bone degradation, and if present, could take steps to slow it (e.g., by local delivery of osteogenic materials (e.g., bone morphogenic proteins or parathyroid hormones)) to build up areas of local bone degradation or weakening or to stop the progression of osteolysis (e.g., by replacing the implant). On the other hand, it is highly undesirable to have to perform invasive surgery in order to evaluate the condition of the implant. Such invasive surgery is not only time consuming, but also costly and painful to the patient. It therefore would be desirable to be able to accurately and non-invasively track wear of the implant, particularly a joint prosthetic, which is subject to generation of wear debris particles.

Currently available detection techniques, however, are crude, invasive, and/or imprecise. There remains tremendous room for improvement in wear detection. It therefore would be desirable to provide a means for monitoring the progression of abrasive wear and debris formation at the interface of two surfaces in contact and moving relative to one another, particularly in biomedical applications, and more particularly in implanted devices without requiring an invasive diagnostic procedure. Desirable, the wear sensing means would not require power sources or microelectronic components as part of the prosthetic implant.

The presence of friction/abrasion at the articulating surfaces of a prosthetic implant also can present difficulties in the delivery of drug at the joint. Common drug delivery systems, including polymer coatings and conventional depots, cannot be used in joints due to the mechanical and abrasive forces present in the joint. It would be advantageous to develop a method of detecting implant or other device wear and/or delivering drug to a joint space.

It would also be desirable to provide new and improved methods and devices for detecting mechanical wear of parts in non-medical applications as well. For example, it would be useful be able to detect wear of moving parts in industrial and automotive applications, such as universal joints, bearings, disk brakes, clutch pads, and other engineered erodible or wear surfaces.

### SUMMARY OF THE INVENTION

In one aspect, a medical implant device is provided which has a mechanical wear detector. The device includes a prosthetic device body having at least one outer surface area; at least one reservoir in the device body; a wear indicator composition disposed in said at least one reservoir, wherein mechanical wear of the at least one outer surface area of the device body in vivo causes release of at least part of the wear indicator composition. The prosthetic device body may be a bone prosthesis or part thereof, such as one adapted for replacement of a hip, a knee, a shoulder, an elbow, or a vertebra.

The device body and surface area (e.g., wear surface) in which the reservoirs are defined typically includes a biocompatible material selected from metals, polymers, ceramics, and combinations thereof. In one instance, the surface area comprises a polyethylene.

In one embodiment, the device includes a plurality of discretely spaced reservoirs, which may be micro-reservoirs. The reservoirs may be formed in the device body by a microfabrication method.

The wear indicator composition may be provided in two or more layers in the reservoir. The wear indicator composition may include one or more matrix materials. For example, the one or more matrix materials may include a biodegradable, water-soluble, or water-swellable matrix material.

In one embodiment, a therapeutic or prophylactic agent may be included with the matrix material, such that when the matrix material degrades (e.g., erodes, biodegrades) or dissolves in vivo the therapeutic or prophylactic agent is controllably released.

In another aspect, an orthopedic implant device is provided for controlled local release of a beneficial substance in vivo. In one embodiment, this device includes a device body which comprises a release system which includes at least one beneficial substance, wherein the beneficial substance is releasable from the device in vivo upon mechanical wear of at least one surface of the device body. In one embodiment, the amount of beneficial substance released is proportional to the amount of mechanical wear experienced by the device body. The beneficial substance may be a therapeutic or prophylactic agent or a biocompatible lubricating agent. The implant device may be part of a knee implant, a hip implant, a bone resurfacing device, or an artificial vertebra.

In one embodiment, the at least one beneficial substance may be disposed in a plurality of discrete reservoirs located in the device body. In another embodiment, the beneficial substance, such as a bisphosphonate drug, may be dispersed in a non-porous polymeric material which forms a wear surface on the device.

In one embodiment, the implant device may include a wear indicator composition in addition to the beneficial substance. For example, the implant device may further include at least one reservoir in the device body, a wear indicator composition disposed in said at least one reservoir, wherein mechanical wear of the at least one outer surface area of the device body in vivo causes release of at least part of the wear indicator composition.

In yet another aspect, a method is provided for non-invasively detecting mechanical wear of a prosthetic device implanted in a human or other animal. In one embodiment, the method comprises the steps of using a non-invasive imaging technique to image the prosthetic device which includes a wear indicating composition, and detecting wear indicating composition release from the prosthetic device. For example, the imaging technique may be selected from magnetic resonance imaging, x-ray, ultrasound, positron emission tomography, or fluoroscopy. In one embodiment, release of the wear indicating composition is detected by identifying the presence of at least a portion of the wear indicating composition at one or more positions remote from its original position in the prosthetic device. In another embodiment, release of the wear indicating composition is detected by identifying the absence of at least a portion of the wear indicating composition from its original position in the prosthetic device.

The prosthetic device may include wear indicating composition that is provided in each of a plurality of discrete reservoirs in the device. The reservoirs may be microreservoirs.

In one embodiment, the method further includes, before the step of using a non-invasive imaging technique, a step of administering to the human or other animal a substance that interacts or binds with the wear indicating composition to enhance the detection of wear indicating composition that has been released from the prosthetic device. For example, the non-invasive imaging technique may be positron emission tomography and the substance may be a radioactive agent.

In still another aspect, a mechanical apparatus is provided that includes a first structure having a wearable surface, which wears upon frictional engagement with a second structure during operation of the apparatus; a plurality of discrete microreservoirs disposed in defined locations in the wearable surface; and at least one wear indicating composition contained in the microreservoirs, wherein upon a predetermined amount of wear of the wearable surface at least a portion of the at least one wear indicating composition is released from one or more of the microreservoirs.

### BRIEF DESCRIPTION OF THE DRAWINGS

FIG. 1A is a perspective view, with partial cut-away, of an acetabulum component of a total hip implant device, showing the metal outer housing and polymeric liner of the component. FIG. 1B is a cross-sectional view of part of the acetabulum component shown in FIG. 1A, illustrating the reservoirs loaded with a wear indicating composition, located in the liner. FIG. 1C illustrates the assembled total hip implant device.

FIG. 2 is a cross-sectional view of one embodiment of a liner for an acetabulum component of a hip implant, showing three discrete, identically dimensioned reservoirs loaded with a wear indicator composition.

FIG. 3 is a cross-sectional view of another embodiment of a liner for an acetabulum component of a hip implant, showing seven discrete, identically dimensioned reservoirs loaded with a wear indicator composition.

FIG. 4 is a cross-sectional view of another embodiment of a liner for an acetabulum component of a hip implant, showing seven discrete reservoirs loaded with a wear indicator composition, with two of the reservoirs being larger in size (extending more deeply into the liner) than the other five reservoirs.

FIG. 5 is a cross-sectional view of another embodiment of a liner for an acetabulum component of a hip implant, showing seven discrete reservoirs loaded with a wear indicator composition, wherein the reservoirs are clustered in different regions of the liner.

FIG. 6 is a cross-sectional view of another embodiment of a liner for an acetabulum component of a hip implant, showing three discrete reservoirs each loaded with a multi-layered wear indicator composition.

FIG. 7A is a cross-sectional view of another embodiment of a liner for an acetabulum component having a reservoir loaded with a wear indicator composition. FIG. 7B is a close-up of the wear indicator composition-loaded reservoir in the same cross-sectional view as in FIG. 7A. FIG. 7C illustrates in a cross-sectional view taken along line C-C three possible embodiments of the shape of the same wear indicator composition-loaded reservoir as in FIG. 7B.

FIG. 8 is a cross-sectional view of another embodiment of a liner for an acetabulum component of a hip implant, showing three discrete reservoirs, two of which are loaded with a multi-layered wear indicator composition and one of which is loaded with a single wear indicator composition and is positioned more deeply within the liner than the other two reservoirs.

FIG. 9 is a cross-sectional view of another embodiment of a liner for an acetabulum component of a hip implant, showing three discrete reservoirs, two of which are loaded with different multi-layered wear indicator compositions and one of which is loaded with a single wear indicator composition, wherein all reservoirs are positioned completely beneath the surface of the liner.

FIG. 10 is a cross-sectional view of another embodiment of a liner for an acetabulum component of a hip implant, showing three discrete reservoirs, each loaded with different multi-layered wear indicator compositions, wherein the reservoirs are positioned with an end opening flush with the surface of the liner.

FIG. 11 is a cross-sectional view of yet another embodiment of a liner for an acetabulum component of a hip implant, wherein the liner includes multiple wear indicator compositions stacked in layers substantially parallel to the surface of the liner.

FIG. 12 is a cross-sectional view of yet another embodiment of a liner for an acetabulum component of a hip implant wherein the liner includes seven discrete, spherical reservoirs located beneath the surface of the liner and each loaded with a wear indicator composition. The two reservoir located near the outer edge of the liner contain a first wear indicator composition and the five reservoirs positioned therebetween contain a second wear indicator composition.

FIG. 13 is a cross-sectional view of yet another embodiment of an acetabulum component liner having seven spherical reservoirs, which are disposed in spaced positions beneath the surface of the liner, and which contain a wear indicator composition.

FIG. 14 is a perspective view of one embodiment of a total knee implant having condyles with reservoirs located therein containing a wear indicating composition.

FIG. 15 is a cross-sectional view of one embodiment of a knee implant, showing a polyethylene component and a tibial component having elongated reservoirs containing three different wear indicator compositions in different reservoirs.

FIG. 16 is a cross-sectional view of another embodiment of a knee implant, showing a polyethylene component and a tibial component having elongated reservoirs containing different wear indicator compositions in different reservoirs, some of which are disposed beneath the interface surface.

FIG. 17 is a cross-sectional view of another embodiment of a knee implant, showing a polyethylene component and a tibial component having elongated reservoirs containing four different wear indicator compositions in different reservoirs, some of which are disposed beneath the interface surface.

FIG. 18 is a cross-sectional view of another embodiment of a knee implant, showing a polyethylene component and a tibial component having elongated and spherical reservoirs containing different wear indicator compositions in different reservoirs.

FIG. 19 is a cross-sectional view of still another embodiment of a knee implant, showing a polyethylene component and a tibial component having elongated and spherical reservoirs containing different wear indicator compositions in different reservoirs.

FIG. 20 is a cross-sectional view, with a close-up view of a portion thereof, of a femoral resurfacing implant device having reservoirs loaded with a wear indicating material or therapeutic agent.

FIGS. 21A-B are cross-sectional views of the device shown in FIG. 20, illustrating in close up an implant device having no mechanical wear and the same device following some mechanical wear in an amount effective to release by abrasion at least a portion of the drug contained in one of the reservoirs.

FIG. 22 is a cross-sectional view of one embodiment of an artificial spinal disk implant having reservoirs loaded with wear indicating composition, the reservoirs being located at the interface of two rotatably engaged plates of the device.

FIG. 23 is a cross-sectional view of another embodiment of a polymeric liner of an acetabular component of a total hip implant, which liner is adapted for drug delivery and is provided with a needle port for in vivo refill.

FIG. 24 is a cross-sectional view of one embodiment of a hip implant device which includes an acetabular cup, a metal femoral ball, and a polyethylene liner in which thin metal foils are embedded to create a series of capacitors for wear measurements.

### DETAILED DESCRIPTION OF THE PREFERRED EMBODIMENTS

Methods and devices have been developed that utilize an abrasion mechanism for the non-invasive detection of medical implant device wear and/or for drug delivery to a joint space. In addition, the methods and devices can be adapted for the detection of device wear or the release of chemicals in non-medical applications, such as the detection of wear or release of molecules in automotive, watercraft, or aircraft parts. The released particles can be a wear indicating material (e.g., a diagnostic agent) or can be a therapeutic or prophylactic agent (e.g., an active pharmaceutical agent or API formulation).

Advantageously, this wear sensor can be “passive,” in the sense that the means for indicating wear requires no electrical or electromechanical component as part of the implant device itself. Release of the indicator material is triggered without electrical power. This beneficially can reduce the cost and complexity of the device, yet can enable the physician to non-invasively monitor the wear of an implanted prosthetic device inside the human or animal patient.

The Wear Indicating Device

In one embodiment, a medical implant device having a mechanical wear detector is provided that includes at least one prosthetic device body having at least one outer surface area; at least one reservoir in the device body; a wear indicator composition disposed in said at least one reservoir, wherein mechanical wear of the at least one outer surface area of the device body in vivo causes, upon a predetermined amount of wear, release of at least part of the wear indicator composition.

As used herein, the term “prosthetic device body” refers to medical and dental devices that are primarily used to secure together separate tissue portions or to provide a load bearing function. It is considered prosthetic in the sense that it is serving as a structural complement or substitute (permanently or temporarily) for one or more tissues of the body, particularly hard tissues.

As used herein, the term “wear indicator composition” refers to a material (e.g., magnetic or non-magnetic particles such as microspheres or nanoparticles, dyes, contrast agents, markers, etc.) that is released from the reservoir by the friction/abrasion/mechanical wearing away of the reservoir contents by movement relative of a contacting surface, which material can be detected non-invasively and is distinguishable from the material(s) of construction of the device body.

In one aspect, the degree of abrasion-induced wear can be correlated noninvasively to a detected presence (or absence) of release particles at one or more locations around the implanted or installed device. This diagnostic agent (i.e., the detected wear indicator composition) is a distinct composition from the “normal” wear debris from the device body. Alternatively or in addition, wear can be measured by detecting the wear indicator composition remaining in the implant reservoir.

In a preferred embodiment, the wear indicator composition (which may include a probe) is incorporated within the device so that it will only be exposed when a particular level of part wear occurs. Multiple probes may be situated in different areas of the device so that different degrees of wear may be measured and their locations determined.

In a preferred embodiment, a reservoir-based, passive sensor is included in the articulating surface of an orthopedic or spinal implant to measure wear of the articulating surface. In a preferred embodiment, as the articulating surface wears, the contents of the reservoir-based sensor are released from the reservoir by abrasion. The wear indicator composition contents of the reservoir (e.g., particles such as microspheres or nanoparticles, dyes, magnetic particles, contrast agents, etc.) are detectable (e.g., the location of the released reservoir contents can be visualized or otherwise determined) by the physician using non-invasive imaging means, such as magnetic resonance imaging (MRI), x-ray, positron emission tomography (PET), ultrasound, fluoroscopy, or other imaging techniques known in the art.

It may be useful or necessary to administer a radioactive substance or other contrast agent to the patient (i.e., implant recipient) to facilitate or enhance imaging. For example, the selected wear indicator composition could be a particle or other material that, following release from the implant, interacts or binds with a radioactive substance prior to a PET scan, to give a significantly larger or smaller signal. Thus, it is the molecule released from the reservoir that serves as a specialized marker for abrasion/wear of the implant device.

The presence of the reservoir contents anywhere outside the reservoir indicates that the material has been released from the reservoir by the friction/abrasion/mechanical wearing away of the reservoir contents by a contacting surface. In another embodiment, different diagnostic materials may be layered within the reservoirs of the device so that the amount of abrasion/wear could be determined by which material and/or how much material has been released from the reservoir. Conversely, instead of using the release of material from the reservoir to determine the amount of wear, the amount of device wear could be determined based on how much material was still left in the reservoir at a given point in time.

As used herein, the phrase “by mechanical wear” refers to and includes release caused by friction, abrasion, other mechanical wearing away of a portion of the device body.

In one embodiment, the wear sensor is in the form of an array of discrete, spaced, reservoirs positioned across one or more (e.g., surface, or subsurface) areas of the device. In another embodiment, the reservoir is in the form of a continuous reservoir in at least one (e.g., surface, or subsurface) areas of the device.

In another preferred embodiment, different materials are placed at specific locations on the implant to allow detection of wear in specific areas. For example, in total knee arthroplasty, the polyethylene component of the artificial knee moves against the metal base plate in the tibia. The degree and direction of this movement is based on the degree to which the knee is flexed. The amount of abrasion, and hence the rate and extent of device wear, will be affected by other factors such as the patient's weight, activity level, and the correct or incorrect positioning of the implant by the physician. To detect wear (in this case, called “backside wear”) at different locations on the polyethylene or the tibial component, the reservoirs contained in either component would be filled with different marker molecules so that detection of a specific compound would indicate wear at a specific location on the device. For example, if red dye were in the right side of the implant and yellow dye were on the left, detection of red dye and absence of yellow dye in the joint space would indicate that the right side of the implant was wearing faster than the left side. This could indicate, for example, sub-optimal placement of the implant or the presence of mechanical forces in the patient (based on how the patient walks, runs, etc.) that were previously undetected in the clinic.

The cement used for fixing some types of orthopedic devices into/onto bone can generate particles that can get caught in the articulating spaces of the artificial joint. Such particles can scratch/abrade the smooth metal, ceramic, or polymer surfaces of the implant device to generate additional particles that can cause osteolysis. In one embodiment, various biocompatible micro/nano particles, contrast agents, dyes, or the like, may be selectively loaded into the cement to allow visualization of cement degradation or cement particle formation. Such patients could be categorized as high risk for osteolysis and monitored more closely than might otherwise be needed.

In one embodiment, a pH dependent dye may be used to detect osteolysis if/when there is an extreme pH change at the site of osteolysis. Similarly, a pH sensor could be incorporated into the implant.

In yet another embodiment, wear could be detected or mitigated by the formation of a material that binds with the particles released or exposed by abrasive wear. For instance, the abrasion may expose reservoir contents containing a material that can bind particles that might be forming as the two implant surfaces rub against one another. For example, the reservoir could be filled with a metal chelating material. If small metal particles are formed during device wear, these metal particles will be bound by the chelating material. Once bound, the metal complex could also have properties that are sufficiently different from the non-bound material, allowing a measurement of the amount of device wear. Such a method may keep the particles from migrating and aggregating at locations susceptible to osteolysis (e.g., the interface between the implant and the bone). In the case of polyethylene particles, a material could be exposed at or released from the reservoirs that selective binds to polyethylene (e.g. a fluorescent or magnetic marker) so that it can be detected. Even if the binding process does not keep the particles from migrating, it will be useful to be able to detect their presence and quantity.

In exemplary non-medical embodiments, the present passive reservoir-based sensors may be included in devices where the failure caused by the abrasion of two surfaces could be costly, time consuming, or catastrophic. For example, such passive monitors could be included in automotive and aerospace parts. In a preferred embodiment, bearings could have small reservoirs embedded below the surface of the bearing. Samples could be taken of fluid in contact with the bearing. If the bearing was worn to the point that the contents of the reservoirs have been released into the fluid by abrasion, testing of the fluid will show the presence of the reservoir contents, indicating that the bearing should be replaced. In a similar embodiment, oil-contacting parts such as pistons could contain such reservoirs and the oil could be tested frequently for the presence of the wear indicating material. Imaging techniques for non-medical applications could include visual imaging with digital cameras and image processing software, fluorescence, ultra-violet light, lasers, resonance techniques, gas or liquid chromatography, or mass spectrometry.

In one embodiment, a combination of different wear indicator compositions is used such that one may determine, by the specific agent detected, the extent of implant wear. For example, in one device, a unique wear indicator composition could be placed in one or more discrete reservoirs at varying “depths” in the implant. In such a design, a shallower reservoir would be exposed first, releasing a first wear indicator material (after a lesser amount of wear has occurred) and then a deeper reservoir would be exposed later, releasing a second wear indicator material (as a greater amount of wear has occurred). A variety of wear indicator compositions are contemplated. In one case, the use of “neutron activation technology” and subsequent detection of the short-lived isotopes that NAT produces could be used, which would permit the detection and identification of the different tracers—if present—that can then be related to the extent of wear. There are commercially available diagnostic assays that could be readily adapted for such embodiments, including the work of the BioPhysics Assay Laboratory, Inc. (BioPAL) (http://www.biopal.com/NA.htm), which has diagnostic assay products which include microspheres containing various lanthanides for detection following neutron activation. In one embodiment, the presence of these products could be monitored (off-line) in fluid samples recovered from the “joint sack” as a way of monitoring wear (and release).

In another aspect, detection of wear is determined indirectly by measuring the resulting osteolysis that can result from the wear debris. In one case, a receptor mediated, contrast assisted diagnostic imaging technology is used. For example, expression (folate) receptors on inflammatory cells associated with (rheumatoid) arthritis could permit the use of folic acid as a vector to target imaging and therapeutic agents to the site of inflammation. Examples of these types of assays have been developed in other areas. For example, Diatide (Londenderry, N.H., now part of Berlex Labs) developed a peptide-Tc 99 conjugate that binds a cellular receptor which is expressed at the site of a blood clot/thrombus. In the present case, a receptor on inflammatory cells associated with osteolysis could permit the use of folic acid (or another agent) as a vector to target imaging of osteolysis and thus wear debris.

In each of the illustrative embodiments described herein, orthopedic applications or orthopedic devices are meant to encompass any devices that are in contact with bone of any kind, including spinal devices such as vertebral fusion devices (e.g., cages, screws, etc.) and artificial discs, maxillofacial reconstruction materials and devices, and any dental devices or prostheses. “Orthopaedic” and “orthopedic” as used herein have the same meaning.

In all medical applications, the devices preferably and importantly are fabricated of biocompatible materials wherever possible. Where it is not possible to use biocompatible materials, then these materials desirably are coated or encapsulated with a biocompatible material, and biological exposure to those components is otherwise minimized. Non-medical applications do not have this limitation, and in such embodiments a wide variety of materials may be used depending upon the particular application.

In order to have the amount of material released from or remaining in the reservoir be proportional to the amount of abrasion experienced by the device, it will be important to match the hardness of the “indictor formulation” (e.g., a probe or marker plus any other materials mixed with or layered with the marker) to the hardness of the surrounding surface. This may not be necessary for applications where the sensor results are to be of the binary (e.g., yes/no) type. For example, in one embodiment, the amount of wear is not important until a critical threshold is reached, and then when the threshold amount of wear is reached, a reservoir may release all of its contents at once, rather than have a gradual release as the indicator formulation abrades with wear.

As used herein, the term “reservoir” can mean discrete locations within the device, or it can indicate a situation where the device surface has a marker or drug uniformly distributed across its surface. Layers containing different markers and/or drugs would be stacked/deposited on one another. As any part of the device surface wears, the marker or drug is released by abrasion. When enough wear has occurred, a new layer of marker or drug is reached and release of that drug or marker begins. Each of these layers can be considered a “reservoir” in that there is a discrete, defined (e.g., pre-selected) location that contains a particular drug or marker. This method will not allow spatial differentiation of wear, but may be easier to deploy than other methods.

In one embodiment, wear is measured as a function of change in concentration of a molecular probe, and the probe will be solubilizable in tissue and/or physiological fluid, as opposed to particulate measurement. The probe will be exposed by a two-step process: abrasion opens the reservoir (e.g., exposes the contents, the layer containing the solid state probe) and the probe dissolves in physiological fluid so that it can be measured. Preferably, a non-invasive test is used to measure the dissolved probe. For example, detection may be via a body scan or by urine sampling. Alternatively, a more invasive but potentially acceptable procedure may be blood sampling.

Selection of the probe would be expected to be based on several requirements. For instance, an appropriate probe should normally be present in negligible quantities or absent in average human physiological fluids and/or tissues, and should be detectable at low concentrations. Multiple probes may be used to differentiate degrees of wear. Examples of probes include metals (e.g., indium), metal compounds e.g., indium nitrate), stable isotopes that do not naturally occur in vivo, small organic molecules (e.g., dyes), and biological molecules (e.g., antibodies).

In one example, the probe may include indium. Indium is non-toxic at low concentrations and is detectable at microgram/L concentrations. Human blood normally contains <1 microgram/L. If a 5-microgram mass of In is distributed in blood, it will be detectable. A microliter reservoir could contain 1000× more In than required for detection, assuming the In is converted to a soluble form and evenly distributed in the body. Urinalysis could be used to monitor levels, using atomic absorption.

In another example, when the metal does not have sufficient solubility, the probe may include water soluble metal salts which can provide a detectable signal. For instance, indium acetate or nitrate are water-soluble and would be efficiently released into physiological fluid.

Small organic molecules could be measured with high sensitivity if they exhibit a spectrum (ultraviolet/visible or fluorescent) that is sufficiently different from other components of physiological fluids and does not have signal interference from components of physiological fluid. Biomolecules could be measured with high sensitivity similarly to small molecules, if they contain a chromophore. Alternatively, a sensitive ELISA could allow quantitation.

To be useful in the present sensor devices, the probe material is fabricated in a physical shape and with properties conducive to a useful rate of release for the purpose of detecting wear. Alternatively, the probe is combined with other ingredients that enhance detectability. Multiple probes may be fabricated and/or formulated uniquely. Formulation could provide better control over release rate after the reservoir has been opened.

The reservoirs also may be designed to release a biocompatible lubricant, in addition to or in place of the wear indicating material. Examples of lubricant materials include silicones, hyaluronan, or hyaluronan-type compounds, gels, and mixtures thereof (e.g., SYNVISC™ (Genyzme Corporation)). The wear indicating material may be selected to also provide some lubrication function, to reduce further wear.

The Drug Delivery Device

In another medical embodiment, the abrasive wear mechanism can be harnessed for controlled drug delivery. In a particular embodiment, the mechanism enables in vivo drug release. In a preferred embodiment, reservoirs containing a drug (anti-inflammatory, growth factor, etc.) and formulation would be present on or near the articulating surface of an artificial joint. As the joint is articulating during use (knee flexion during walking), abrasion causes the drug to be released from the reservoir. The rate and amount of drug release will be proportional to the use of the joint, so more active individuals will receive proportionately more drug than less active individuals. In a preferred embodiment, the patient has had a portion of the distal end of their femur (at the knee joint) re-surfaced and the re-surfaced portion is in contact with and is articulating against the cartilage of the proximal end of the tibia (at the knee joint). As the knee is used, the re-surfaced surface rubs against the cartilage. The abrasion of the two surfaces will cause drug contained in reservoirs in the re-surfaced surface to release growth factors promoting cartilage growth/repair such as FGF, IGF, and TGF-β. Because the rate and amount of drug release is proportional to use of the joint, more active individuals will get more growth factor to grow/repair cartilage that has been exposed to more wear and tear (i.e., like a “passive feedback mechanism” that releases more drug only to those that need it).

In one embodiment, an orthopedic implant device, such as a knee or hip prosthesis is provided for controlled local delivery in vivo of one or more drugs. This is particularly useful for certain drugs and/or certain patients where systemic delivery poses unacceptable risks or side effects. In a particularly preferred embodiment, the implant device includes a bisphosphonate compound, and the bisphosphonate is provided in the device in such a way that release is controlled and occurs essentially only in response to and proportionally to in vivo mechanical wear of the implanted device, effectively operating as a passive biofeedback system. The more wear that is occurring, the more drug is released in response. Ideally, the device will be tailored to deliver an appropriate amount of the drug to negate the amount of osteolysis that would be expected to occur based on the amount of wear debris generated. In one embodiment, the bisphosphonate is dispersed in a polymeric material which forms a wear surface on the device. For example, the bisphosphonate could be loaded homogeneously throughout a polyethylene liner. The bisphosphonate should be trapped within the polymeric material so as not to leach out before the polymer matrix wears down to expose a surface that includes bisphosphonate molecules. That is, the polymeric material should be non-porous. Preferably, the bisphosphonate is homogeneously dispersed in the non-porous polymeric matrix material. In another embodiment, the bisphosphonate is loaded in one or more discrete enclosed reservoirs in the body of the device, or a part thereof, such as a polymeric (e.g., a polyethylene) liner. In still another embodiment, the bisphosphonate can be incorporated (e.g., dispersed) into a bone cement that is used to secure the prosthetic implant in vivo. As used herein, the term “bisphosphonate” refers to analogues of pyrophosphate that are involved in calcium homeostasis. Representative examples of bisphosphonates include pamidronate, zoledronic acid, residronate, alendronate, pamidronate, clodronate, tetrasodium pyrophosphate, incadronate, minodronate, olpadronate, ibandronate, etidronate, and tiludronate.

Sometimes cemented implants can become loose during in vivo use when the interface between the implant and the cement fails. (One researcher recently reported that over 55% of failed hip replacements were caused by component loosening.) In one embodiment, the implant releases one or more growth factors, such as a bone morphogenic protein (BMP), as the loosened hip moves against the cement. The BMP desirably would travel out of the ends of the cemented zone and possibly could cause bone to grow in the space between the cement and the loosened implant, re-fixing the implant in place and eliminating the need for a total hip arthroplasty (THA). Such release of growth factors like BMP would be controlled, at least in part, by the amount of movement between the implant and the cement. If there is substantial motion, there is a greater potential for the generation of cement particles that may travel to the end of the hip stem and cause severe osteolysis. If BMP is released with this motion-induced rubbing, the BMP can stimulate bone growth in the same area that the cement particles collect. In this way, an osteogenic factor may be provided to counteract the osteolytic factor.

These devices can be used deliver a range of different drugs depending upon the particular application. In one embodiment, the drug is used in the management of pain and swelling following the implantation surgery. For example, the device can release an effective amount of an analgesic agent alone or in combination with an anesthetic agent. In another embodiment, the drug helps minimize the risk of prosthetic joint infection or other site-specific infection due to implantation of an orthopedic or dental device. For example, the device can release a therapeutic or prophylactic effective amount one or more antibiotics (e.g., cefazolin, cephalosporin, etc.) and/or another agent effective in preventing or mitigating biofilms (e.g., a quorum-sensing blocker or other agent targeting biofilm integrity). Bacteria tend to form biofilms on the surface of implant devices, and these biofilms, which are essentially a microbial ecosystem with a protective barrier, are relatively impermeable to antibiotics. Accordingly, systemically administered antibiotics may not achieve optimal dosing where it is needed most. However, the present devices enable the delivery of the desired dose of antibiotic precisely when and precisely where needed—in particular beneath the biofilm. In addition, the device can be designed to release the drug in various temporal and spatial patterns/profiles, e.g., releasing drug in a continuous or pulsatile manner for several (e.g., 5 to 15) days and/or targeting areas of the device, if any, that are more conducive to bacterial growth.

In one embodiment, the present drug-eluting device is adapted for use in the treatment of cancer of the bone or joint. For example, osteosarcoma or chondrosarcoma often are treated surgically by excision requiring removal of significant amounts of bone and soft tissue. Care must be taken to avoid spilling the tumor during resection to avoid seeding of tumor cells into surrounding tissues. It therefore would be beneficial for the prosthetic implant to release one or more local chemotherapeutic agents into the surrounding tissue following implantation, in order to destroy tumor cells remaining at the surgical site following resection, to complement or replace the systemic chemotherapy and/or radiation therapy that typically is prescribed for the patient. In variations of these embodiments, the implant device releases one or a combination of therapeutic agents, including chemotherapeutic agents (e.g., paclitaxel, vincristine, ifosfamide, dacttinomycin, doxorubicin, cyclophosphamide, and the like), bisphosphonates (e.g., pamidronate, clodronate, zoledronic acid, and ibandronic acid), analgesics (such as opoids and NSAIDS), anesthetics (e.g., ketoamine, bupivacaine and ropivacaine), tramadol, and dexamethasone.

In another embodiment, the drug facilitates vascularization at or into the implanted prosthetic device or promotes bone health and growth. For example, the drug can be a bone morphogenic protein (BMP) or recombinant version thereof (rBMP), which facilitates bone formation around or, in the case of a device having a porous surface, into the implanted prosthetic device. Examples of BMPs include BMP-2, -3, -4, -7, and -9, where rhBMP-2 may be preferred. This could be particularly desirable where the prosthesis is secured without the use of cement, although it could possibly be used in combination with a cement.

The device may release a combination of different substances to improve bone healing. For example, the device can release different combinations of growth factors (e.g., (TGF)-β, BMP, VEGF), osteoinductive molecules, hormones, anti-TNF (tumor necrosis factor) agents, and bone-forming cells (e.g., osteoblasts, adult stem cells, osteoprogenitor cells). These different molecules and cells can be delivered at varied spatial positions and temporal sequences during bone healing. In one embodiment for the repair of local bone erosions, which often are associated with rheumatoid arthritis, the prosthetic device locally delivers (1) an anti-TNF agent, which reduces inflammation that fuels bone erosion, and (2) parathyroid hormone (PTH), which stimulates bone formation, and/or osteoprotegrin (OPG), which blocks bone resorption and can lead to repair of local bone erosions and reversal of systemic bone loss. Examples of anti-TNF agents include TNF antagonists, such as etanercept (Enbrel™, Amgen and Wyeth) and infliximab (Remicade™, Centocor), which have shown efficacy and have been approved by the U.S. FDA for the treatment of rheumatoid arthritis.

In yet another embodiment, the drug can be one selected to mitigate the risk of formation of blood clots at the implant site, which can lead to venous thromboembolism or pulmonary embolism. For instance, the device may be used to release one or more anticoagulants and/or antiplatelet drugs (e.g., heparins, aspirin, clopidogrel, lepirudin, fondaparinux, warfarins, dicumarol, etc.).

In still a further embodiment, the drug stored in and released from the reservoirs is a self-propagating agent, such as a gene therapy agent or vector. A desired local or systemic response is created following release of the small amount of agent.

Representative examples of therapeutic or prophylactic agents that may be released from the prosthetic device include analgesics, anesthetics, antimicrobial agents, antibodies, anticoagulants, antifibrinolytic agents, antiinflammatory agents, antiparasitic agents, antiviral agents, cytokines, cytotoxins or cell proliferation inhibiting agents, chemotherapeutic agents, hormones, interferons, and combinations thereof. In one embodiment, the device provides for the controlled release of a growth factor, such fibroblast growth factors, platelet-derived growth factors, insulin-like growth factors, epidermal growth factors, transforming growth factors, cartilage-inducing factors, osteoid-inducing factors, osteogenin and other bone growth factors, and collagen growth factors. In another embodiment, the device provides for controlled release of a neutrophic factor (which may be of benefit in spinal prosthetic applications) or a neutrophic factor.

In one embodiment, the drug is in an encapsulated form. For example, the drug can be provided in microspheres or liposomes for sustained release.

In one aspect, an implantable prosthetic device for controlled drug delivery is provided which includes: a prosthetic device body having at least one outer surface area expected to be subjected to abrasion following implantation; one or more defined reservoirs located in within the body; a release system disposed in the reservoirs which comprises at least one therapeutic or prophylactic agent, wherein following implantation into a patient the therapeutic or prophylactic agent is released by abrasive wear of the release system and/or by abrasive wear of a region of the device body disposed between an outer surface and the release system.

Illustrative Embodiments of Implants Having Passive Wear Sensors

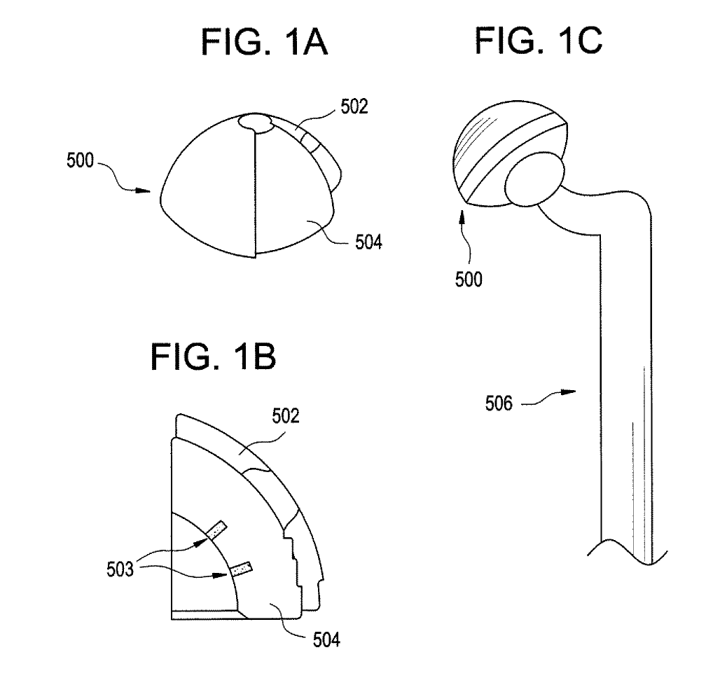

FIGS. 1A-C illustrate a total hip implant device that includes acetabulum component 500 and stem component 506. The acetabulum component includes a metal outer housing 502 and a polyethylene line 504. The liner includes a plurality of discrete reservoirs 503 (two are shown) which are loaded with a wear indicating composition. The reservoirs may be microreservoirs.

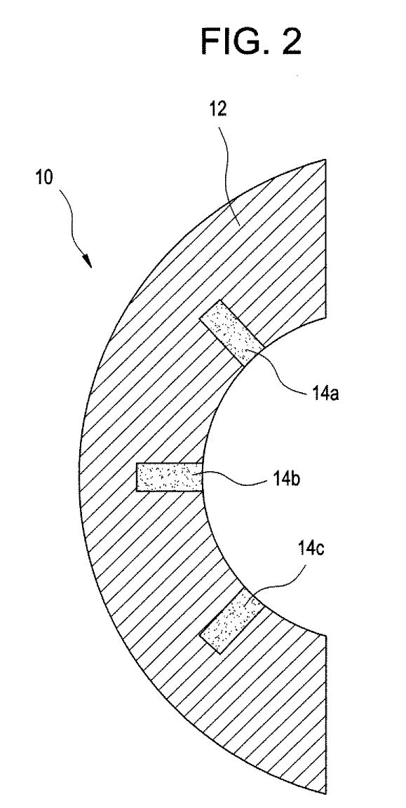

FIGS. 2-13 are cross-sectional views of a variety of possible embodiments of liners of an acetabular component of a total hip implant. The liner may be a polyethylene material or other suitably lubricious biocompatible polymer, metal, ceramic, or other material. Other variations and combinations of these embodiments are envisioned.

FIG. 2 shows device 10 that includes liner 12 having three reservoirs 14a-c disposed in the liner 12 in spaced apart positions. One end of each of the reservoirs is open to the concave surface of the liner, which interfaces with the femoral ball (not shown). The discrete reservoirs 14a-c are elongated (e.g., cylindrical) and are filled with a wear indicator composition (e.g., a probe or a marker material).

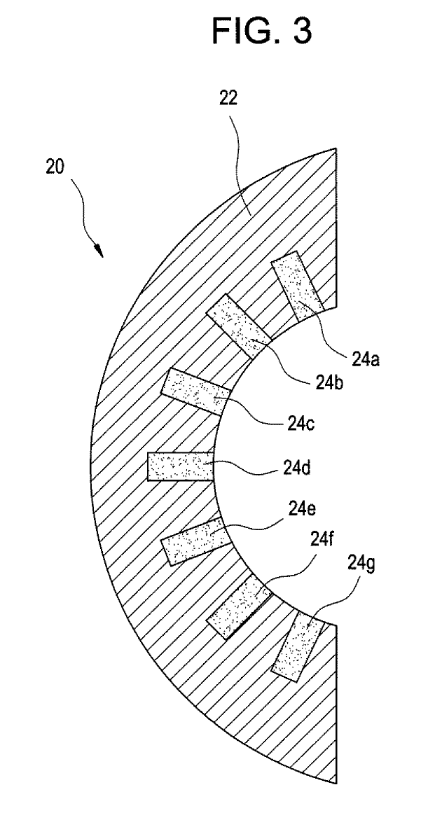

FIG. 3 shows device 20 that includes liner 22 having seven reservoirs 24a-g disposed in the liner 22 in spaced apart positions. One end of each of the reservoirs is open to the concave surface of the liner, which interfaces with the femoral ball (not shown). The reservoirs 24a-g are elongated (e.g., cylindrical) and are filled with a wear indicator composition (e.g., a probe or a marker material).

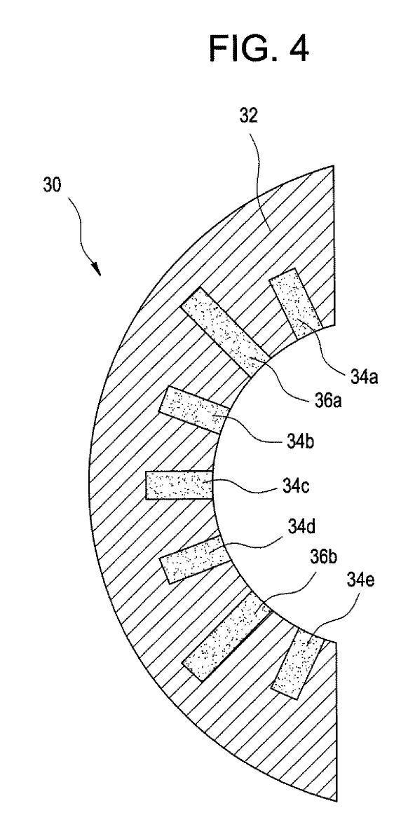

FIG. 4 shows device 30 that includes liner 32 having seven reservoirs 34a-e and 36a-b disposed in the liner 32 in spaced apart positions. One end of each of the reservoirs is open to the concave surface of the liner, which interfaces with the femoral ball (not shown). The reservoirs 34a-e are relatively shallow compared with reservoirs 36a-b, which are relatively deeper and extend almost through the liner. All of the reservoirs are filled with the same wear indicator composition (e.g., a probe or a marker material).

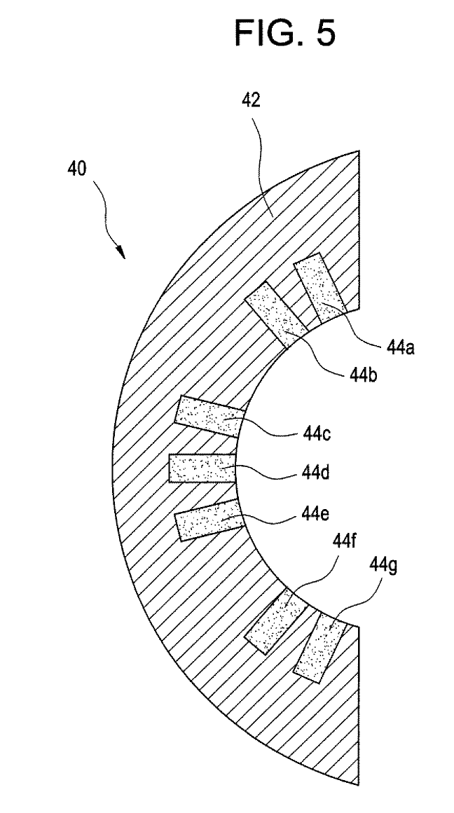

FIG. 5 shows device 40 that includes liner 42 having seven reservoirs 44a-g disposed in the liner 42 in spaced apart positions. One end of each of the reservoirs is open to the concave surface of the liner, which interfaces with the femoral ball (not shown). The reservoirs 44a-g are elongated (e.g., cylindrical) and are filled with a wear indicator composition (e.g., a probe or a marker material). The reservoirs are clustered in different in particular regions of the liner. FIGS. 2, 3, and 5 illustrate that the number and spacing of the reservoirs can be varied, e.g., to provide different degrees of wear sensor coverage in the device.

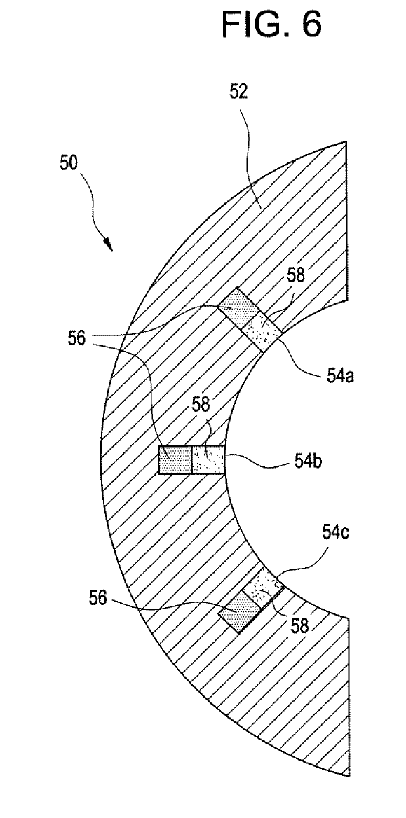

FIG. 6 shows device 50 that includes liner 52 having three reservoirs 54a-c disposed in the liner 52 in spaced apart positions. One end of each of the reservoirs is open to the concave surface of the liner, which interfaces with the femoral ball (not shown). The elongated reservoirs 54a-c are each filled with a multi-layered wear indicator composition (e.g., a probe or a marker material). In particular, the composition includes a first composition forming outer layer 58 and a second composition forming inner layer 56. In use, the detection of released composition 56 shows that substantial wear has occurred, approaching the midpoint of the liner at least one point.

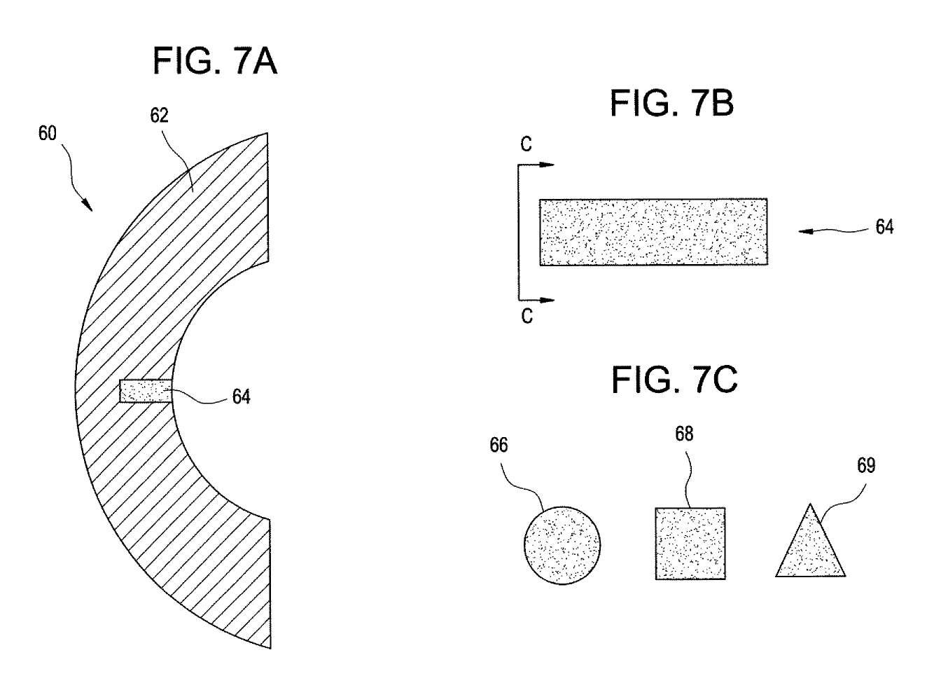

FIGS. 7A-C show device 60 that includes liner 62 having a single reservoir 64 disposed in the liner 62 at the apex. One end of the reservoir is open to the concave surface of the liner, which interfaces with the femoral ball (not shown). The elongated reservoir 64 is filled with a wear indicator composition. FIG. 7C shows that the (other) cross-sectional shape of the reservoir can be varied, such as circular, square, or triangular.

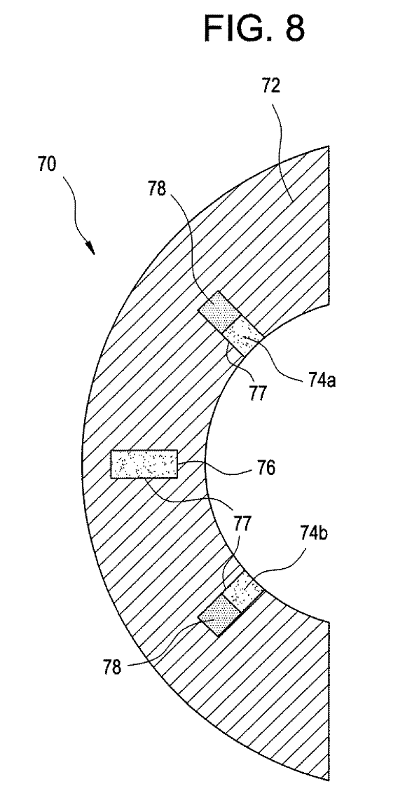

FIG. 8 shows device 70 that includes liner 72 having three reservoirs 74a-b and 76 disposed in the liner 72 in spaced apart positions. One end of each of reservoirs 74a-b is open to the concave surface of the liner, which interfaces with the femoral ball (not shown), but reservoir 76 is disposed within the liner beneath the concave surface of the liner. Elongated reservoirs 74a-b are each filled with a multi-layered wear indicator composition which includes a first composition forming outer layer 77 and a second composition forming inner layer 78. Elongated reservoir 76, which is located between reservoirs 74a and 74b, is filled with first composition 77.

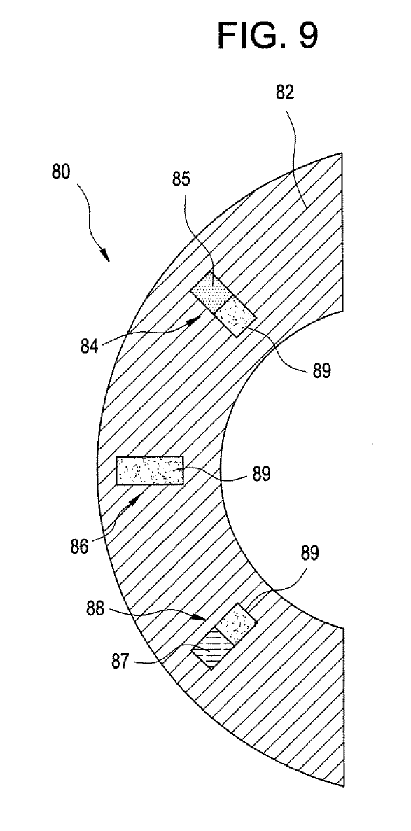

FIG. 9 shows device 80 that includes liner 82 having three elongated reservoirs 84, 86, and 88 disposed in the liner 82 in spaced apart positions imbedded in the liner. One end of each reservoir is beneath the concave surface of the liner, and the other end is beneath the convex surface of the liner. Reservoir 84 is filled with a multi-layered wear indicator composition which includes a first composition forming outer layer 89 and a second composition forming inner layer 85. Reservoir 88 is filled with a multi-layered wear indicator composition that includes a first composition forming outer layer 89 and a third composition forming inner layer 87. Reservoir 86, which is located between reservoirs 84 and 88, is filled with first composition 89.

FIGS. 8-10 illustrate at least one highly useful aspect of the invention: Both the composition and location of the reservoirs can be varied to include select combinations different wear indicator materials and/or different reservoir locations, in order to be able to determine not just the presence of wear, but the degree and location of that wear, based for example on the particular wear indicator material or combination of wear indicator materials detected (or absent). For instance, the degree of wear can be indicated by using multiple, different indicator materials at different depths, so that the particular indicator materials detected, or not detected, shows that the device has, or has not, worn to a particular depth. Similarly, by using multiple, different indicator materials at different regions of the device, one can determine where the wear has occurred.

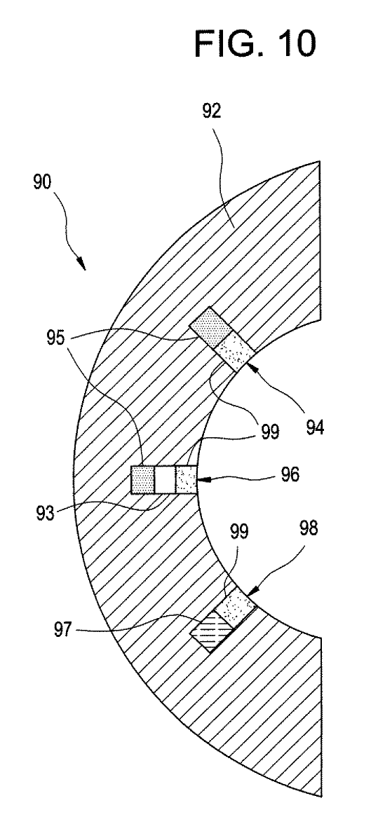

FIG. 10 shows device 90 that includes liner 92 having three reservoirs 94, 96, and 98 disposed in the liner 92 in spaced apart positions. One end of each of the reservoirs 94, 96, and 98 is open to the concave surface of the liner, which interfaces with the femoral ball (not shown). Each elongated reservoir is filled with a multi-layered wear indicator composition, which includes a first composition forming outer layer 99. However, each reservoir includes an inner layer or layers that are different from one another: Reservoir 94 includes a second composition forming inner layer 95, reservoir 98 includes a third composition forming inner layer 97, and reservoir 96 includes a middle layer 93 of a fourth composition and an inner layer of second composition 95.

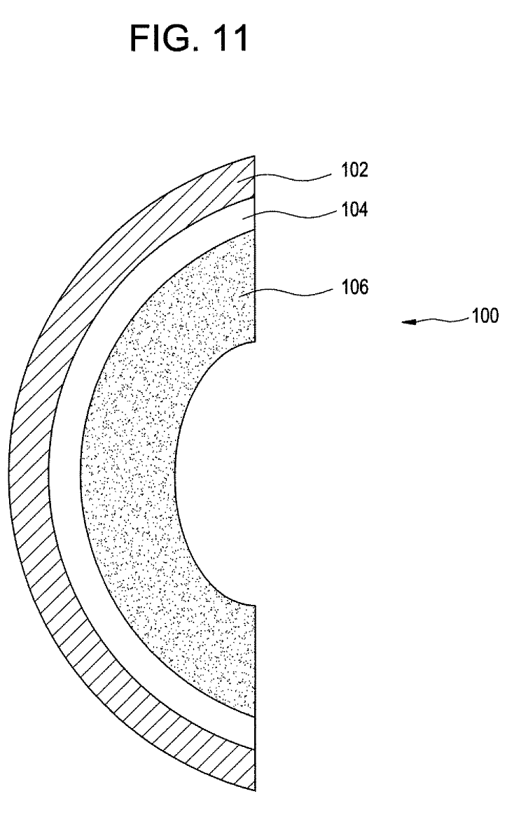

FIG. 11 shows device 100 that includes a liner having an outer layer 102, a middle layer 104, and an inner layer 106. The out layer is polyethylene, and the inner and middle layers are reservoirs that are loaded with different wear indicator compositions. In an alternative embodiment, only the middle layer 104 contains the wear indicator composition, and the inner layer 106 is also a polyethylene.

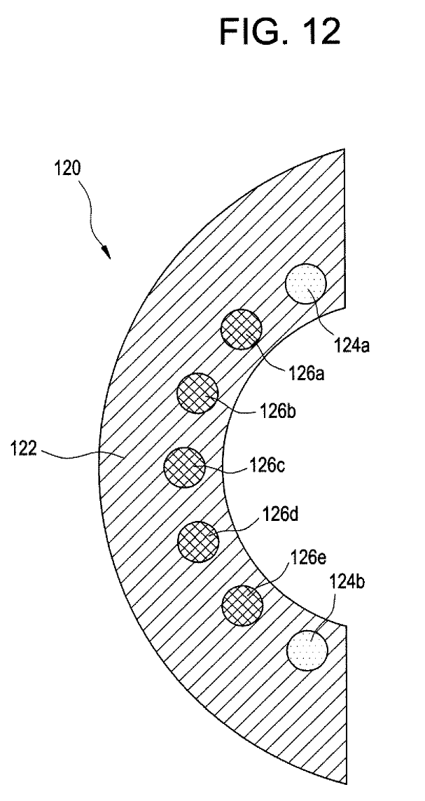

FIG. 12 shows device 120 that includes liner 122 having seven reservoirs 124a-b and 126a-c imbedded in the liner 122 in spaced apart positions beneath the concave surface of the liner. In contrast to the reservoirs of FIGS. 2-11, these reservoirs are spherical. Reservoirs 124a and 124b are located near the outer edge of the liner, with reservoirs 126a-e positioned therebetween. Reservoirs 124a-b contain a first wear indicator composition, and reservoirs 126a-e contain a second wear indicator composition. In use, the physician may be able to determine where on the implant the abrasion/wear is occurring, based upon which wear indicator composition(s) is/are detected.

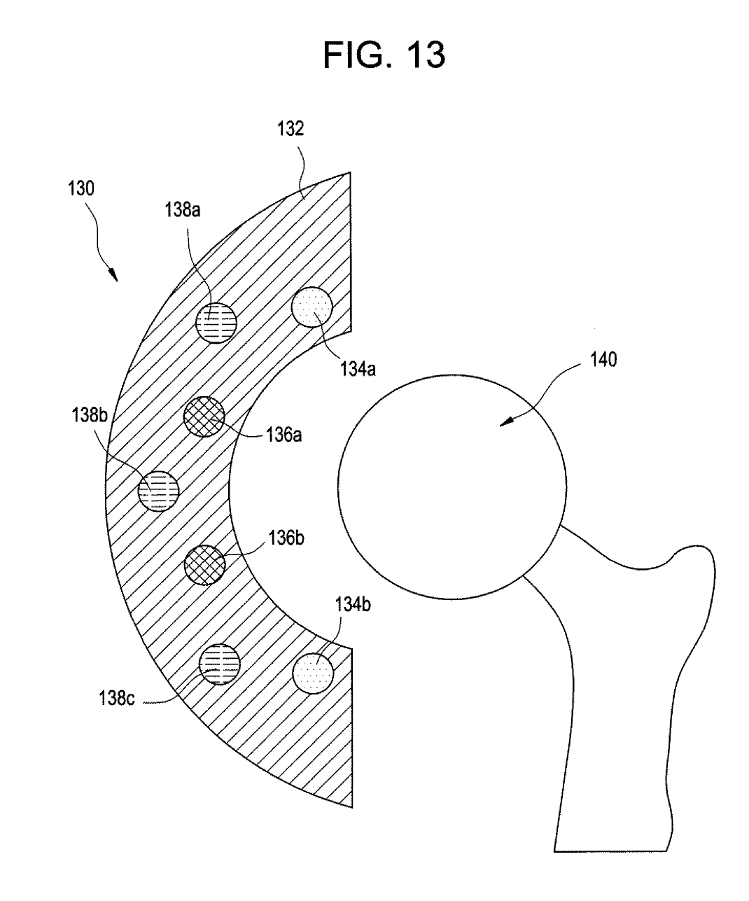

FIG. 13 shows device 130 that includes liner 132 having seven spherical reservoirs 134a-b, 136a-b, and 138a-c imbedded in the liner 132 in spaced apart positions beneath the concave surface of the liner. Reservoirs 134a and 134b are located near the outer edge of the liner, with reservoirs 136a-b positioned therebetween at the same depth beneath the inner surface of the liner as reservoirs 134a-b. Reservoirs 138a-c are located in the spaces between the other reservoirs, but at a greater depth beneath the inner surface of the liner than the other reservoirs. Reservoirs 134a-b contain a first wear indicator composition, reservoirs 136a-b contain a second wear indicator composition, and reservoirs 138a-c contain a third wear indicator composition. In use, the physician may be able to determine where on the implant the abrasion/wear is occurring and how deeply the liner has worn, based upon which wear indicator composition(s) is/are detected. Device 130 would be used for example in conjunction with a femoral component of a total hip implant, wherein the ball 140 of the femoral component fittingly engages with liner 132.

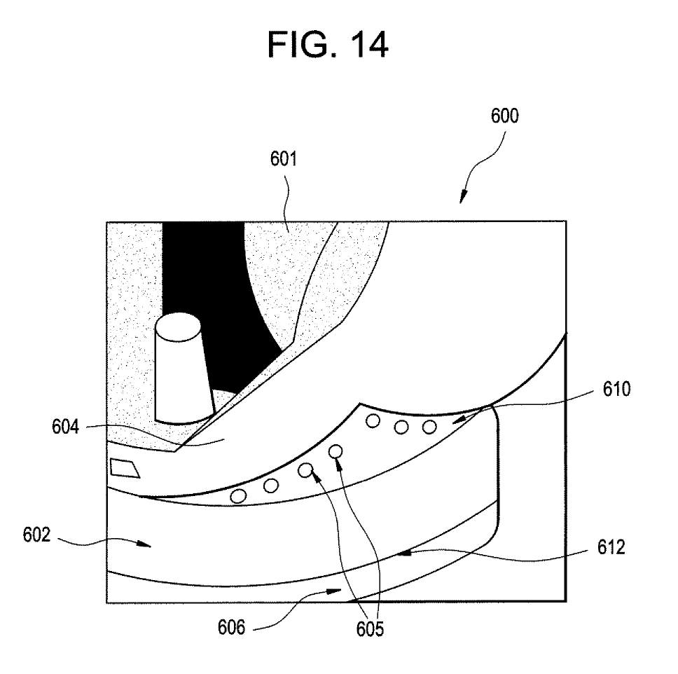

FIGS. 14-19 are cross-sectional views of a variety of possible embodiments of polyethylene component and the tibial component of a total knee implant. Other variations and combinations of these embodiments are envisioned.

FIG. 14 shows a total knee implant 600. The device includes condyles 604 of the femoral component 601 of the implant. The condyles engage, or interface, with a contoured polyethylene component 602 of a tibial component 606 of the implant. Tibial component 606 and femoral component 601, which typical include a metal and/or ceramic featured for securement with and integration into existing bone tissue. Reservoirs 605 are shown located in component 604, which contain at least one wear indicating composition. (Reservoirs containing a wear indicating composition alternatively or additionally may be located in component 602.) Wear and particle generation can occur at interface 610. In addition, “backside wear” and particle generation can occur at interface 612.

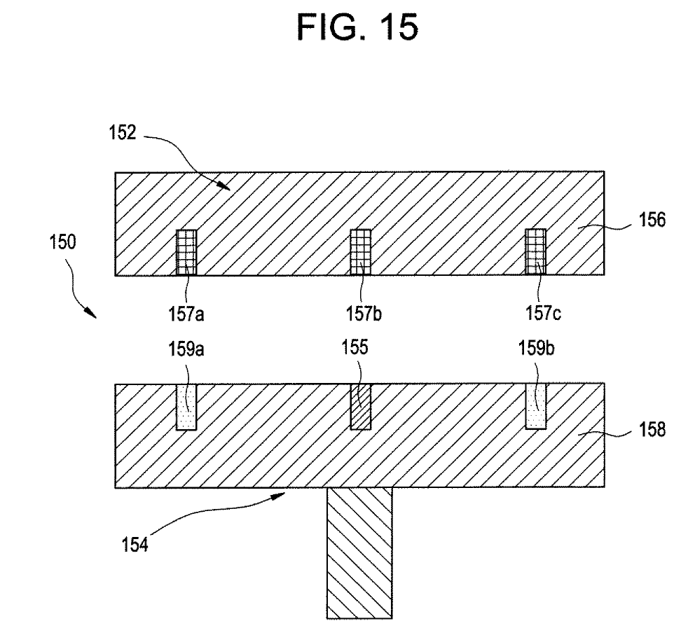

FIG. 15 shows implant device 150, which includes polyethylene component 152 and tibial component 154. Component 152 includes a device body 156 in which three elongated reservoirs 157a-c are located in spaced apart positions, with one end of each of the reservoirs open to the interface surface, which interfaces with component 154. The reservoirs 157a-c are filled with a first wear indicator composition (e.g., a probe or a marker material). Component 154 includes a device body 158 in which three elongated reservoirs 155 and 159a-b are located in spaced apart positions, with one end of each of the reservoirs open to the interface surface, which interfaces with component 152. The reservoirs 159a-b are filled with a second wear indicator composition (e.g., a probe or a marker material), and reservoir 155 is filled with yet a third wear indicator composition.

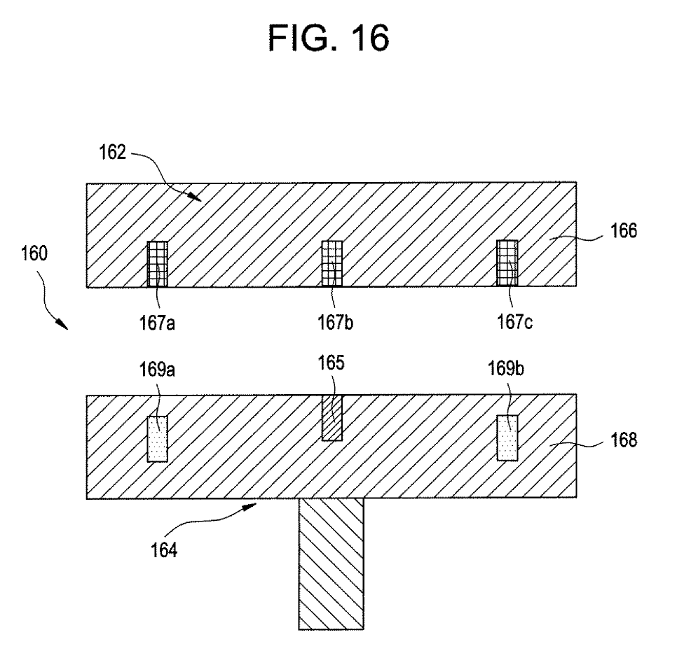

FIG. 16 shows implant device 160, which includes polyethylene component 162 and tibial component 164. Component 162 includes a device body 166 in which three elongated reservoirs 167a-c are located in spaced apart positions, with one end of each of the reservoirs open to the interface surface, which interfaces with component 164. The reservoirs 167a-c are filled with a first wear indicator composition (e.g., a probe or a marker material). Component 164 includes a device body 168 in which three elongated reservoirs 165 and 169a-b are located in spaced apart positions, with one end of reservoir 165 open to the interface surface. Reservoirs 169a-b are, however, imbedded beneath the interface surface. The reservoirs 169a-b are filled with a second wear indicator composition (e.g., a probe or a marker material), and reservoir 165 is filled with yet a third wear indicator composition.

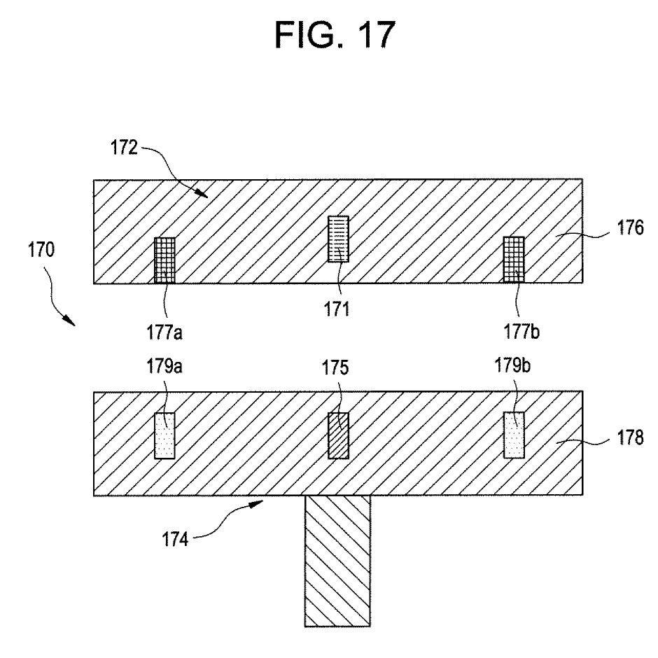

FIG. 17 shows implant device 170, which includes polyethylene component 172 and tibial component 174. Component 172 includes a device body 176 in which three elongated reservoirs 177a-b are located in spaced apart positions, with one end of each of the reservoirs open to the interface surface, which interfaces with component 174, and elongated reservoir 171 imbedded beneath the interface surface. The reservoirs 177a-b are filled with a first wear indicator composition, and reservoir 171 is filled with a second wear indicator composition. Component 174 includes a device body 178 in which three elongated reservoirs 175 and 179a-b are located in spaced apart positions, imbedded beneath the interface surface. The reservoirs 179a-b are filled with a third wear indicator composition, and reservoir 175 is filled with yet a fourth wear indicator composition.

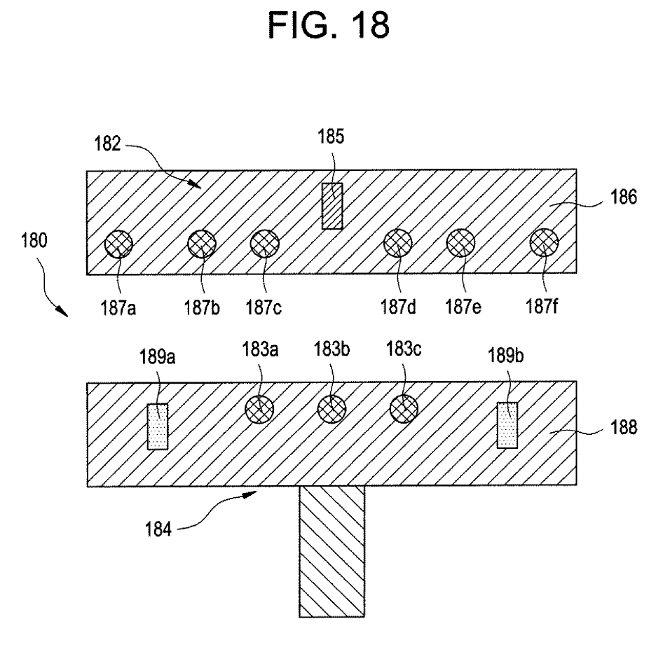

FIG. 18 shows implant device 180, which includes polyethylene component 182 and tibial component 184. Component 182 includes a device body 186 having a centrally located, deeply embedded reservoir 185, loaded with a first wear indicator composition, and having spherical reservoirs 187a-f located in spaced apart positions, shallowly imbedded beneath the interface surface, which are loaded with a second wear indicator composition. Component 184 includes a device body 188 having two elongated, imbedded reservoirs 189a-b and three spherical reservoirs 183a-c located in spaced apart positions between reservoirs 189a-b and imbedded beneath the interface surface. The reservoirs 189a-b are filled with a third wear indicator composition, and reservoirs 183a-c are filled with yet a fourth wear indicator composition.

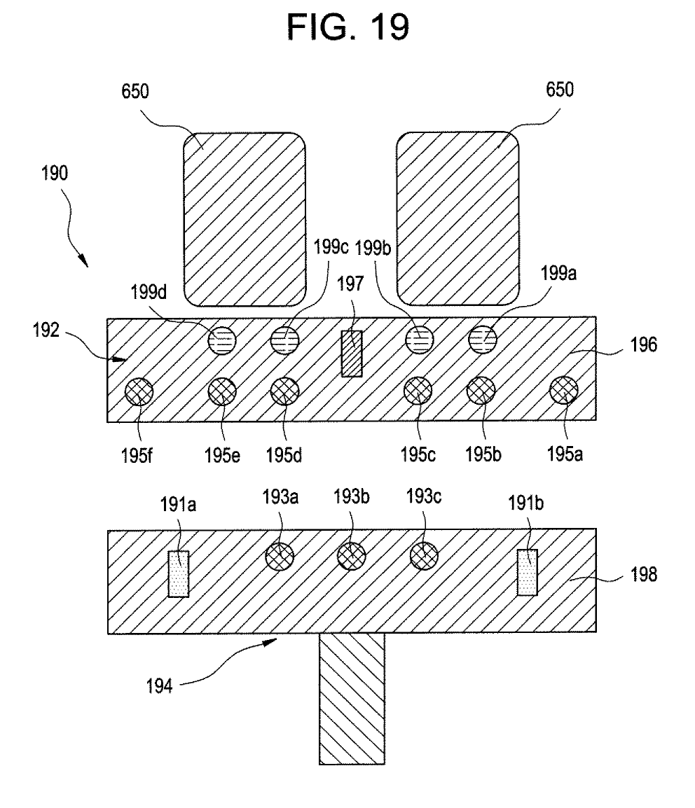

FIG. 19 shows implant device 190, which includes polyethylene component 192 and tibial component 194. Condyles 650 of the femoral component of a total knee implant are also shown. Component 192 includes a device body 196 having a centrally located, elongated embedded reservoir 197, loaded with a first wear indicator composition; six spherical reservoirs 195a-f located in spaced apart positions, shallowly imbedded beneath the interface surface, loaded with a second wear indicator composition; and four spherical reservoirs 199a-d located in spaced apart positions, deeply imbedded beneath the interface surface, loaded with a third wear indicator composition. Component 194 includes a device body 198 having two elongated, imbedded reservoirs 191a-b and three spherical reservoirs 193a-c located in spaced apart positions between reservoirs 191a-b and imbedded beneath the interface surface. The reservoirs 191a-b are filled with a third wear indicator composition, and reservoirs 193a-c are filled with the second wear indicator composition. Other variations and combinations of these illustrated embodiments are envisioned.

In the various medical implant embodiments described herein in which the liner is described as being a polyethylene, it is understood that the polyethylene is one known in the art to be suitable for biomedical implants generally and for a wear surface material in particular. It is also understood that any suitable polymeric material other than a polyethylene is contemplated for use in the devices and methods described herein.

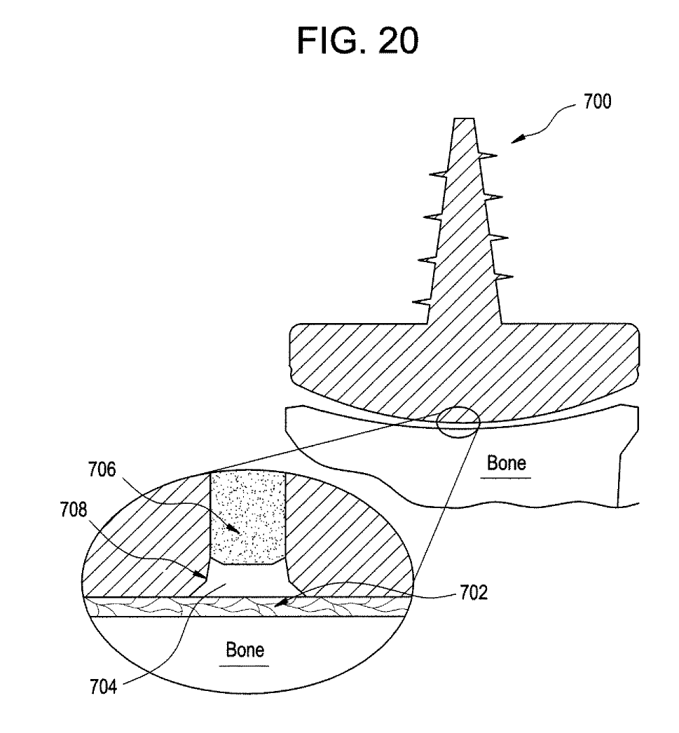

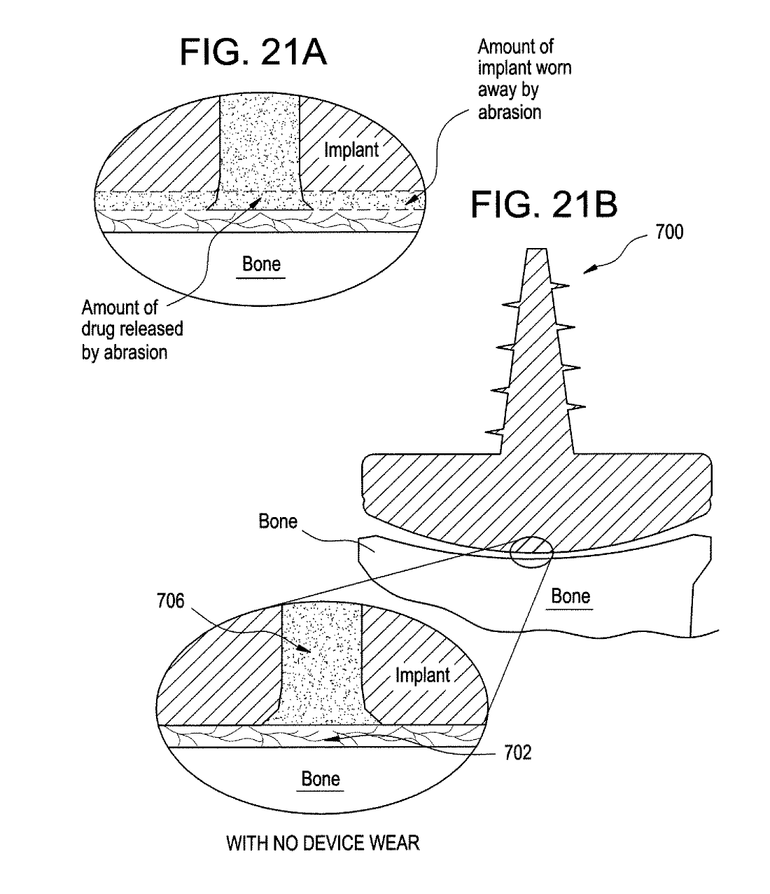

FIG. 20 shows a bone resurfacing implant device 700 having reservoirs 704, which are loaded with a material 706, which may be a wear indicating substance and/or one or more therapeutic agents (such as a growth factor to promote chondrogenesis. The reservoirs can protect growth factors from damage at articular surfaces with the objective of extending the longevity of a resurfaced joint. The openings 708 of the reservoirs 704 that interface with intra-articular cartilage 702 desirably have smooth rounded edges. In an exemplary embodiment, such a resurfacing implant device is adapted for resurfacing of a femur. Such resurfacing devices could also be used in the knee and shoulder, among other joint surfaces. FIGS. 21A-B show an embodiment of the implant device having no mechanical wear and the same device following some mechanical wear in an amount effective to release by abrasion at least a portion of drug contained in one of the reservoirs.

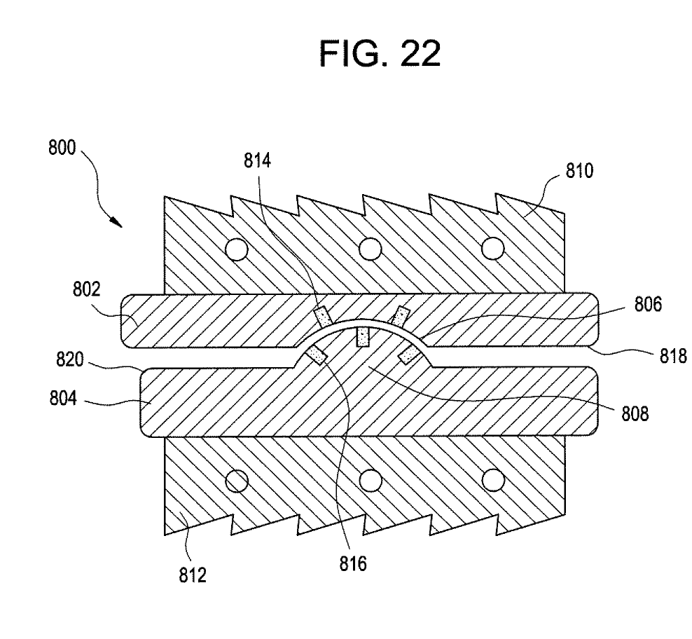

FIG. 22 shows an artificial spinal disk device 800 having an upper end plate 802 and a lower end plate 804. The upper and lower plates include upper keel 810 and lower keep 812, respectively. The plates interface and rotate about hemispherically shaped articulating element 808 rising from surface 820 of lower plate 804. The articulating element 808 rotatably engages into hemispherically shaped cavity 806 in surface 818 of upper plate 802. As this interface is where wear particle generation may occur, the surface (or subsurface) of the cavity 806 includes reservoirs 814 which contain a first wear indicating composition. The surface of articulating element 808 includes reservoirs 816 which contain a second wear indicating composition. In different embodiments, the number, size, location, shape, degree of filling, and content of the reservoirs can be varied in the artificial spinal disk device. For example, those variations may be like those describe above for hip or knee implant devices. Reservoir may be provided in only the upper plate or only in the lower plate. Additional reservoirs may be provided on one or both of the keels to release therapeutic agents, such as antibiotics growth factors, etc.

In yet another aspect, the wear surfaces of the implant device may include reservoirs that are intended to capture any particles that find their way into the space between moving surfaces, e.g., between the ball and socket of a joint, thereby preventing the particles from creating more wear in the joint. This may be accomplished by filling a reservoir with a soft biocompatible gel, into which the rogue particles can become imbedded. The distance between the structural wear surface and the surface of the gel (which desirably is below the wear surface) may be varied among different reservoirs in a single device in order to capture different sized particles (assuming most particles that are produced as a result of wear are approximately spherical). The diameter of the reservoir opening also may be varied to facilitate capture of different sized particles. These capture-reservoirs may also release detectable compounds when the surface of the gel is disrupted. The compounds may be different in each reservoir, so that a physician can determine the size range of the particles in the joint as well as the location of the wear within the joint.

Illustrative Embodiment of Implant Having Active Wear Sensors

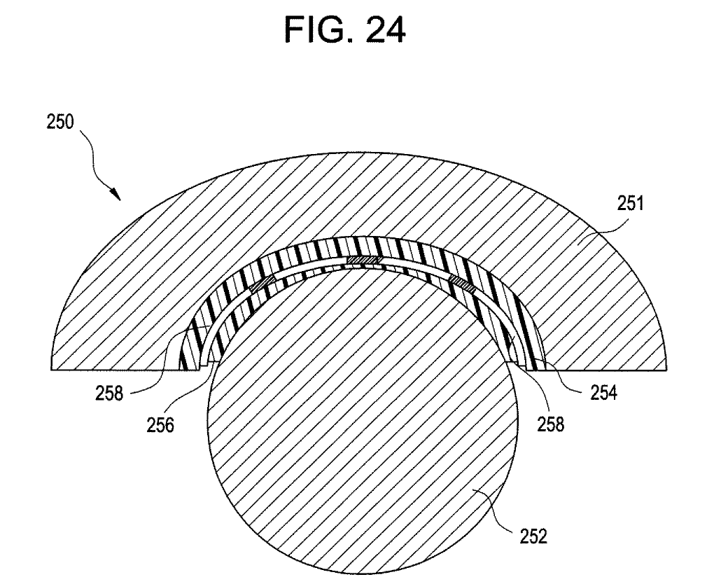

In another aspect, an active wear sensor is provided in the prosthetic implant. One embodiment of such a hip implant device is shown in FIG. 24.

Device 250 includes acetabular cup 251, metal femoral ball 252, and polyethylene liner 254/256. In the liner, thin metal foils 258 are embedded, to create a series of capacitors for wear measurements. In one approach, capacitors may be combined with thin film inductors to create antennas whose frequency is correlated to the wear. Measurements also could be made using microneedles to make direct contact or non-invasively using AC external fields.

Illustrative Embodiments of the Drug-Eluting Prosthesis

The abrasion mechanism of controlled drug release described above may be used in the delivery of a variety of drugs from prosthetic devices, alone or in combination with the passive wear sensors described above.

The “prosthetic” device body is a medical device primarily used to secure together separate tissue portions. It is considered “prosthetic” in the sense that it is serving as a structural complement or substitute for one or more tissues of the body. For example, in one embodiment, the device body is a surgical staple or a surgical screw. The staple or screw is provided with a plurality of microreservoirs that store and release drug. In one embodiment, the staple or screw is biodegradable and releases the drug in a defined manner as the screw or staple degrades. In another embodiment, the screw or staple is non-biodegradable, and the plurality of microreservoirs located in the surface of the screw or staple release drug in a defined manner, as dictated by the particular drug formulation contained in the reservoirs. Representative examples of screws and staples that could be modified to include drug containing and releasing reservoirs are described in U.S. Pat. No. 5,961,521 to Roger, which is expressly incorporated herein by reference.

FIG. 20 illustrates another embodiment of an arthrosurface implant having a reservoir containing growth factor for passive, local controlled release in vivo to a bone or joint surface. FIGS. 21A-B illustrate embodiments arthrosurface implant for passive release of drug from a reservoir, with FIG. 21B showing the device before any wear has occurred and FIG. 21A showing the device where sufficient abrasion of the device has occurred to expose/release the drug formulation.

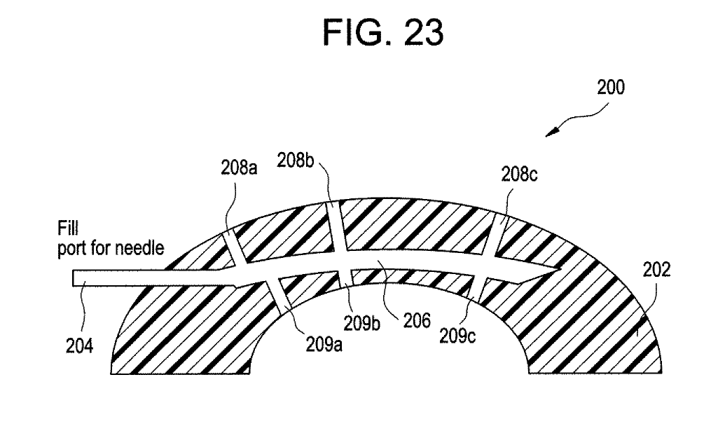

In another aspect, a drug delivery implant is provided that can be refillable in vivo. For example, as shown in FIG. 23, the polyethylene liner of an acetabular component of a total hip implant may be provided with a needle port. Device 200 includes liner 202 in which conduits 208a-c and 209a-c are disposed, having openings in both the convex (fixation) surface and concave (wear) surface, respectively. The other ends of these conduits are open to central depot 206, which is refillable via needle port 204. In an alternative embodiment, conduits 208a-c could extend from a first depot and conduits 209a-c could extend from a second, different depot, such that different materials could be released from each side of the device and could be refilled separately. For example, a therapeutic, such as a BMP could be released to the fixation side, and lubricants could be released to the wear side. Other variations and combinations of these embodiments are envisioned.

Additional Device Details and Methods of Use

Device Body

In one embodiment, the prosthetic device body is a joint or bone prosthesis or part thereof. Examples of typical prosthetic joints include knees, hips, shoulders, and to a lesser extent, elbow, wrist, ankle, and finger joints. In a preferred embodiment, the bone prosthesis is adapted for use in a knee replacement or a hip replacement. The hip is essentially a ball and socket joint, linking the “ball” at the head of the thigh bone (femur) with the cup-shaped “socket” in the pelvic bone. A total hip prosthesis is surgically implanted to replace the damaged bone within the hip joint. In one example, the total hip prosthesis consists of three parts: (1) a cup that replaces the hip socket, which cup is typically polymeric, but also may be ceramic or metal; (2) a metal or ceramic ball that replaces the damaged head of the femur; and (3) a metal stem that is attached to the shaft of the bone to add stability to the prosthesis. The reservoirs can be provided on any or all of the outer surfaces of such a prosthesis. In one embodiment, a stem portion of the prosthesis has an outer surface which includes drug-containing reservoirs.

In other embodiments, the bone prosthesis is adapted for a knee, a shoulder, an elbow, a spinal disk, a dental implant, or a urethral prosthesis. In one embodiment, the device is a spinal disk prosthesis. For example, it could be an adaptation of, or similar to, the FDA-approved CHARITEÉ™ disk (made by DePuy Spine, Inc., of Raynham, Mass.), which comprises cobalt chromium endplates and an Ultra-High Molecular Weight Polyethylene (UHMWPE) sliding core. In one example, the endplates are provided with an array of discrete reservoirs in one or more surfaces, which are loaded with a release system comprising one or more therapeutic or prophylactic agents for controlled release. In another embodiment, the device is a spinal infusion device, such as a modification of the INFUSE® Bone Graft/LT-CAGE Lumbar Tapered Fusion Device (Medtronic Inc.), which is indicated for spinal fusion procedures in skeletally mature patients with degenerative disc disease (DDD). In one modification, the device body, or cage, that holds the rBMP-soaked sponge, is itself provided a plurality of reservoirs, for releasing one or more bioactive agents, to enhance to effectiveness of the device. For instance, the reservoirs could release additional rBMP, antibiotics, analgesics, anesthetics, or combinations thereof. In another variation, the cage device is modified so that the separate rBMP-soaked sponge is no longer needed, thereby greatly simplifying the device preparation steps preceding implantation. For example, the cage device itself can be modified to include reservoirs on the inside and/or outside walls of the cage. These reservoirs contain and passively release an rBMP formulation. As for providing a tissue scaffold or other osteoconductive material inside the cage, the interior can include a dry hydrogel coating material. The surgeon simply wets the coating with saline prior to implantation of the device—no longer need to prepare solution, soak the sponges, and then insert the sponges into the cage. Furthermore, the interior of the cage can be made to have a series of baffles to provide additional surface area for bone growth and/or additional surface area for drug-containing reservoirs.

In another embodiment, the device is for disk and vertebral replacement. For example, the device can be an artificial disk similar to the MAVERICK™ (Medtronic Sofamor Danek) artificial disc for use in patients who suffer from degenerative disc disease. In a further embodiment, the device is used in the treatment of ankylosing spondylitis, a rheumatic disease characterized by inflammation of joints and ligaments, which results in bone erosion, most often in the spine but sometimes in other joints too. The formation of new bone during healing can lead to the fusing of vertebrae and spine rigidity. The device preferably is provided with a plurality of discrete reservoirs, which can be located for example in screws of the device and in surfaces contacting the vertebrae. Such reservoirs could be loaded with a stable OP-1 formulation with optimised release kinetics and optionally loaded with an antibiotic agent for biofilm control. These or other reservoirs could be sized and located to enhance device fixation, e.g., by promoting osteointegration.

In still other embodiments, the device is a dental or maxillofacial prosthetic device. In a preferred variation, the reservoirs of the device release one or more anti-infective agents.

In preferred embodiments, the device body and surface area in which the reservoirs are defined can be formed of, be coated with, or otherwise comprise a biocompatible material selected from metals, polymers, ceramics, and combinations thereof. Typically, the device body is non-biodegradable, as the prosthetic device is intended to last for an extended period of time, preferably for the life of the patient. For instance, the device body can comprises a stainless steel, a chrome-cobalt alloy, a titanium alloy, a ceramic, or an ultra high molecular weight polyethylene. In other embodiments, the device body is formed of or includes a ceramic (e.g., alumina, silicon nitride), a semiconductor (e.g., silicon), a glass (e.g., Pyrex™, BPSG), or a degradable or non-degradable polymer.

The surface of the device body where the reservoirs are located can be porous or non-porous. Optimal bony-ingrowth is expected to be provided into prosthesis devices that include pores of approximately 250 to 500 microns. In one embodiment, the entire surface of the device is porous. In another embodiment, a portion, e.g., a portion of the tissue- or bone-mating surfaces, of the prosthesis is porous, to provide at least one tissue-contact surface that provides stable fixation in the body. The device may include various combinations of porous and non-porous substrate (body) materials with different reservoirs. For example, a portion of the device body may have a non-porous region with a porous surface region in which discrete reservoirs are disposed in spaced positions (i.e., in an array). The reservoirs are filled with drug formulation, such as drug dispersed in a soluble or biodegradable matrix material, such as biocompatible polymer, e.g., PLGA or PEG. In this embodiment, the reservoirs are located only in the porous region. Alternatively, the reservoirs may extend into the non-porous region. Some reservoirs may be shallower or deeper than others, such that only the deeper ones extend into the non-porous region. In such an embodiment, the shallower reservoirs contain a first drug formulation, and the deeper reservoirs are filled with two or more distinct layers: An outer layer, which can be formed of one or more non-bioactive materials (e.g., a biodegradable, protective reservoir cap) that can delay exposure of an inner layer, which can comprise a drug—the same as or different from the drug in formulation. A surface may comprise both porous and non-porous regions. The non-porous region may include reservoirs containing a drug formulation, and the porous region may, for example, be selected to have a porosity that facilitates tissue ingrowth. Other variations and combinations of these embodiments are envisioned.

Optionally, the device body may be installed into the bone site with a biocompatible cement. The surface of the device body to be cemented can be porous or non-porous. The shape of the device body depends on the particular application. The device body preferably is a rigid, non-degradable structure. The body may consist of only one material, or may be a composite or multi-laminate material that is, composed of several layers of the same or different substrate materials that are bonded together. In another embodiment, the device body is not actually a prosthetic but is used in the treatment of an orthopedic disease or disorder.

Reservoirs

The reservoir is located in predefined positions within the device body. The reservoirs are not random or interconnected pores. In one embodiment, the reservoirs are formed with an opening at the surface of the device body and extend into the device body. In other embodiments, the reservoirs are disposed beneath an outer surface of the device body. In one embodiment, a plurality of discrete reservoirs is disposed in an array throughout one or more regions (or areas) of the device body.

Reservoirs can be created in the device body simultaneously with formation of the device body, or it can be made formed in the device body after the device body is made. Accordingly, the reservoirs can be made by a variety of techniques, including MEMS fabrication processes, microfabrication processes, or other micromachining processes, various drilling techniques (e.g., laser, mechanical, and ultrasonic drilling), and build-up or lamination techniques, such as LTCC (low temperature co-fired ceramics). Numerous other methods known in the art can also be used to form the reservoirs. See, for example, U.S. Pat. No. 6,123,861 and U.S. Pat. No. 6,808,522. Microfabrication methods include lithography and etching, injection molding and hot embossing, electroforming/electroplating, microdrilling (e.g., laser drilling), micromilling, electrical discharge machining (EDM), photopolymerization, surface micromachining, high-aspect ratio methods (e.g., LIGA), micro stereo lithography, silicon micromachining, rapid prototyping, and DEEMO (Dry Etching, Electroplating, Molding).

The reservoirs can be fabricated into the device body by any of a number of methods and techniques known in the art, depending on various parameters including the materials of construction of the device body, the dimensions of the reservoirs, the location of the reservoirs on the device body, and the shape and configuration of the device body. In one embodiment, the reservoirs are formed in the substrate by laser drilling, EDM, or other mechanical or physical ablative methods. In another embodiment, the reservoirs are fabricated by a masking and chemical etching process. In embodiments where the device comprises a porous surface, the reservoirs can be fabricated before or after a porosity-inducing step. For instance, reservoirs can be mechanically formed into the porous surface, optionally penetrating into the non-porous region beneath. Alternatively, porosity can be creating in the surface, for example, by a chemical etching process after formation of the reservoirs. In order to preserve the defined boundaries of the reservoirs, the reservoirs can be filled with a temporary fill material, such as a wax, that is resistant to the chemical etch, prior to etching and then the fill material can be removed following etching, for example, by heating and volatilizing the wax or by use of an appropriate solvent selective for the temporary fill material. One process for creating surface microporosity in a titanium or other metal surface is described in U.S. Patent Application Publication No. 2003/0108659 A1 to Bales et al., which is incorporated herein by reference.

In one embodiment, the device includes a plurality of microreservoirs. In drug deliver applications, arrays of discrete microreservoirs may be preferred. A “microreservoir” is a reservoir having a volume equal to or less than 500 μL (e.g., less than 250 μL, less than 100 μL, less than 50 μL, less than 25 μL, less than 10 μL, etc.) and greater than about 1 nL (e.g., greater than 5 nL, greater than 10 nL, greater than about 25 nL, greater than about 50 nL, greater than about 1 μL, etc.). In certain embodiments, microreservoirs are preferred, e.g., to minimize changes to the mechanical integrity of the device (i.e., to avoid negatively impacting the device's ability to withstand the substantial mechanical forces (which can be a multiple of the implant patient's weight) experienced by the prosthetic during use. Microreservoirs also may be preferred to minimize the quantity of indicator material held, thereby avoiding concentrations in vivo that might trigger negative tissue reactions in vivo.

In another embodiment, the reservoirs are macroreservoirs. A “macroreservoir” is a reservoir having a volume greater than 500 μL (e.g., greater than 600 μL, greater than 750 μL, greater than 900 μL, greater than 1 mL, etc.) and less than 5 mL (e.g., less than 4 mL, less than 3 mL, less than 2 mL, less than 1 mL, etc.). The shape and dimensions of the reservoir, as well as the number of reservoirs, can be selected to control the contact area between the drug material and the surrounding surface of the reservoirs. Unless explicitly indicated to be limited to either micro- or macro-scale volumes/quantities, the term “reservoir” is intended to encompass both.

In one embodiment, the wear indicator material is loaded into the device by an ion implantation process, which processes are known in the art in connection with making products outside the field of medical implants. The ion, which may for example be boron or phosphorus, advantageously can be implanted into a device body at or below the wear surface as an add on manufacturing step of a pre-existing manufacturing process for making the device body, rather than requiring a completely new or substantially reconfigured manufacturing process.

Release System and Therapeutic/Prophylactic Agent

The release system comprises at least one therapeutic or prophylactic agent (sometimes referred to herein as a “drug”). The release system is disposed in the reservoirs, so as to be isolated, e.g., protected, from the environment outside of the reservoir until a selected point in time, when its release or exposure is desired. The therapeutic or prophylactic agent can be dispersed in a matrix material, which by its degradation, dissolution, or diffusion properties provides a means for controlling the release kinetics of the therapeutic or prophylactic agent. See, e.g., U.S. Pat. No. 5,797,898.

The therapeutic or prophylactic agent can be essentially any active pharmaceutical ingredient or API. It can be natural or synthetic, organic or inorganic molecules or mixtures thereof. The therapeutic or prophylactic agent molecules can be mixed with other materials to control or enhance the rate and/or time of release from an opened reservoir.

The therapeutic or prophylactic agent molecules may be in essentially any form, such as a pure solid or liquid, a gel or hydrogel, a solution, an emulsion, a slurry, or a suspension. In various embodiments, the therapeutic or prophylactic agent molecules may be in the form of solid mixtures, including amorphous and crystalline mixed powders, monolithic solid mixtures, lyophilized powders, and solid interpenetrating networks. In other embodiments, the molecules are in liquid-comprising forms, such as solutions, emulsions, colloidal suspensions, slurries, or gel mixtures such as hydrogels.

In a preferred embodiment, the drug is provided in a solid form, particularly for purposes of maintaining or extending the stability of the drug over a commercially and medically useful time, e.g., during storage in a drug delivery device until the drug needs to be administered. The solid drug matrix may be in pure form or in the form of solid particles of another material in which the drug is contained, suspended, or dispersed. In one embodiment, the drug is formulated with an excipient material that is useful for accelerating release, e.g., a water-swellable material that can aid in pushing the drug out of the reservoir and through any tissue capsule over the reservoir.

In one embodiment, the drug is formulated with one or more excipients that facilitate transport through tissue capsules. Examples of such excipients include solvents such as DMSO or collagen- or fibrin-degrading enzymes.

The drug can comprise small molecules, large (i.e., macro-) molecules, or a combination thereof. In one embodiment, the large molecule drug is a protein or a peptide. In various other embodiments, the drug can be selected from amino acids, vaccines, antiviral agents, gene delivery vectors, interleukin inhibitors, immunomodulators, neurotropic factors, neuroprotective agents, antineoplastic agents, chemotherapeutic agents, polysaccharides, anti-coagulants (e.g., LMWH, pentasaccharides), antibiotics (e.g., immunosuppressants), analgesic agents, and vitamins. In one embodiment, the drug is a protein. Examples of suitable types of proteins include, glycoproteins, enzymes (e.g., proteolytic enzymes), hormones or other analogs (e.g., LHRH, steroids, corticosteroids, growth factors), antibodies (e.g., anti-VEGF antibodies, tumor necrosis factor inhibitors), cytokines (e.g., α-, β-, or γ-interferons), interleukins (e.g., IL-2, IL-10), and diabetes/obesity-related therapeutics (e.g., insulin, exenatide, PYY, GLP-1 and its analogs). In one embodiment, the drug is a gonadotropin-releasing (LHRH) hormone analog, such as leuprolide. In another exemplary embodiment, the drug comprises parathyroid hormone, such as a human parathyroid hormone or its analogs, e.g., hPTH(1-84) or hPTH(1-34). In a further embodiment, the drug is selected from nucleosides, nucleotides, and analogs and conjugates thereof. In yet another embodiment, the drug comprises a peptide with natriuretic activity, such as atrial natriuretic peptide (ANP), B-type (or brain) natriuretic peptide (BNP), C-type natriuretic peptide (CNP), or dendroaspis natriuretic peptide (DNP). In still another embodiment, the drug is selected from diuretics, vasodilators, inotropic agents, anti-arrhythmic agents, Ca⁺ channel blocking agents, anti-adrenergics/sympatholytics, and renin angiotensin system antagonists. In one embodiment, the drug is a VEGF inhibitor, VEGF antibody, VEGF antibody fragment, or another anti-angiogenic agent. In yet a further embodiment the drug is a prostaglandin, a prostacyclin, or another drug effective in the treatment of peripheral vascular disease.

In still another embodiment, the drug is an angiogenic agent, such as VEGF. In a further embodiment, the drug is an anti-inflammatory, such as dexamethasone. In one embodiment, a device includes both angiogenic agents and anti-inflammatory agents.

The reservoirs in one device can include a single drug or a combination of two or more drugs, and can further include one or more pharmaceutically acceptable carriers. Two or more drugs can be stored together and released from the same one or more reservoirs or they can each be stored in and released from different reservoirs.

The release system may include one or more pharmaceutical excipients. The release system may provide a temporally modulated release profile (e.g., pulsatile release) when time variation in plasma levels is desired or a more continuous or consistent release profile when a constant plasma level as needed to enhance a therapeutic effect, for example. Pulsatile release can be achieved from an individual reservoir, from a plurality of reservoirs, or a combination thereof. For example, where each reservoir provides only a single pulse, multiple pulses (i.e., pulsatile release) are achieved by temporally staggering the single pulse release from each of several reservoirs. Alternatively, multiple pulses can be achieved from a single reservoir by incorporating several layers of a release system and other materials into a single reservoir. Continuous release can be achieved by incorporating a release system that degrades, dissolves, or allows diffusion of molecules through it over an extended period. In one embodiment, the drug formulation within a reservoir comprises layers of drug and non-drug material. After the active release mechanism has exposed the reservoir contents, the multiple layers provide multiple pulses of drug release due to intervening layers of non-drug. Such a strategy can be used to obtain complex release profiles.

Reservoir Caps

In an optional embodiment, the device further includes reservoir caps. A reservoir cap is a discrete structure (e.g., a membrane or thin film) positioned over or disposed in (thereby blocking) the opening of a reservoir to separate the (other) contents of the reservoir from the environment outside of the reservoir. It controls, alone or in combination with the release system, the time and/or rate of release of the therapeutic or prophylactic agent from the reservoir. For example, release can be controlled by selecting which reservoir caps, how many reservoir caps, and where the reservoir caps are located in the device body, how thick the reservoir caps are, and how easily or quickly the reservoir cap will rupture by abrasion to expose the release system/reservoir contents.

In a preferred embodiment for drug delivery, the reservoir cap is non-porous and bioerodible, or capable of being abraded away to initiate release of the drug-containing reservoir contents.

In one embodiment, a discrete reservoir cap completely covers a single reservoir opening. In another embodiment, a discrete reservoir cap covers two or more, but less than all, of the reservoir's openings.

In various embodiments, the reservoir caps may be formed from a material or mixture of materials that degrade, dissolve, or disintegrate over time, or that do not degrade dissolve, or disintegrate, but are permeable or become permeable to the therapeutic or prophylactic agent. Representative examples of reservoir cap materials include polymeric materials, and non-polymeric materials such as porous forms of metals, semiconductors, and ceramics. Passive semiconductor barrier layer materials include nanoporous or microporous silicon membranes.

Cartilage Engineering

In another aspect, implant devices are provided to promote the growth of avascular tissue, such as articular cartilage, and extend the longevity of a person's natural cartilage—e.g., to delay the need for a total knee replacement. In one embodiment, a reservoir-containing drug delivery device is placed in or near the intercondylar fossa, between the condyle, or within/under the synovial sac, and the reservoirs of the device are loaded with a formulation for controlled release of one or more growth factors (FGF, IGF, TGF-β, etc.) to promote chondrogenesis. The device body (substrate) can be shaped and sized to fit near, and provide local drug release to, the cartilage without interfering with movement of the joint.

In another embodiment, devices and methods are provided for use in joint resurfacing. For example, in a conventional resurfacing system, a metal cap is placed over the end of an articular surface to extend the useful life of a failing joint. The present improvement provides a cap having a plurality of discrete reservoirs for releasing growth factors or other therapeutic agents to promote chondrogenesis. In one embodiment, the device includes a body portion and reservoirs, which are loaded with a release system that includes a growth factor. The reservoirs have openings that have smooth rounded edges to minimize frictional engagement with the surface of the adjacent cartilage.

In another embodiment, following a total knee replacement, the prosthetic knee device includes a plurality of discrete reservoirs for releasing an antibiotic or other drug.

Sterilization

When forming all or part of an implantable medical device, the device must be made sterilize. Sterility of the final product is required to render the device suitable for implantation into a human or other patient. This applies to the reservoirs in a metal orthopaedic implant or other prosthetic implant (as described herein), as well as to other kinds of multi-reservoir devices for controlled release of drugs or diagnostic agents or for controlled exposure of sensors and other subcomponents.

The sterilization processes described herein may be applicable various components of different reservoir-based devices for controlled exposure of reservoir contents. Accordingly, sterilization of a “device” or “component” as described herein may encompass microchips, catheters, stents, pumps, polymer matrices, sensors, substrate portions, housings/packaging, orthopaedic/spine/dental devices, and the like. Sterility assurance should be considered in the design of the device's assembly process.

The face of the device will be in direct contact with the body and must be sterile at the time of implant. The reservoir contents will be exposed to the body during the normal course of operation of the device. This will require that the interior surfaces of the reservoir be sterile and that the reservoir contents (e.g., drug or biosensor) be sterile also.

Sterilization processes may differ depending upon whether the implant device or component thereof includes passive or active electronic circuits. As used herein, “passive electronic circuits” refers to the fact that there are no transistors integrated into the silicon. Passive electronic components (e.g., resistors, capacitors, diodes) do not require a power source to operate. In contrast, active electronic components (e.g., transistors) do require a power source.

Drug Delivery Devices

1. Sterilizing the Reservoir Device Body

In one embodiment, the device body includes a silicon chip containing passive electronic circuits. A glass layer may be bonded to the silicon to increase the volume of the reservoirs. Devices with passive circuits can be sterilized by a variety of means, including ethylene oxide (ETO), dry or steam heat, and radiation methods. Typical materials used to construct the device body (crystalline silicon, metals, ceramics) are relatively impervious and will not absorb the ETO like polymeric materials. The relatively low temperatures and limited durations required for dry heat or steam sterilization are unlikely to result in any thermally-induced changes to the device body (e.g., morphology of metal reservoir caps). Similarly, at the relatively low doses of gamma radiation required for sterilization, one generally would not expect to alter thin metal films or alloys, such as may be used to form the reservoir caps.

In embodiments in which a silicon substrate may incorporate active components such as transistors, ETO sterilization should also be suitable for these solid-state devices. The times and temperatures of dry heat and steam sterilization will not be sufficient to alter the electronic performance characteristics of the devices. However, radiation sterilization methods such as gamma or electron-beam irradiation generally should be avoided to prevent damage to devices with circuitry containing transistors, particularly CMOS circuits. Nevertheless, there may be certain instances (e.g., with bipolar transistors) where gamma or electron-beam sterilization is suitable for active components.

2. Sterile Loading of the Drug Formulation

The drug formulation must be prepared and introduced into the reservoir device in a sterile manner. Processes and procedures used in the pharmaceutical industry for the preparation of sterile drug formulations may need to be adapted for the smaller reservoir volumes and more complex formulations used. For example, the two-part formulation of leuprolide used in an in vivo (dog) study (Nature Biotechnology, 24:437-38 (April 2006)) involved lyophilization of a sterile-filtered solution of leuprolide. The sterile filtrate was introduced into the microchip reservoirs using an aseptic filling process. The lyophilizate was then infiltrated with a gamma-sterilized polyethylene glycol, again introduced into the reservoirs using an aseptic filling process.

3. Sterile Sealing of the Reservoirs

After the drug formulation has been introduced into the reservoirs of the substrate/device body, then the reservoirs must be sealed. The sealing operation must be conducted in a sterile environment using sterile materials and aseptic technique. A variety of materials may be used to form the seal, including low-temperature solders, silicon or ceramic “chips”, metal rings and grooves (as described in US 2006/0115323 A1), or other materials (e.g., polymer tape for nonhermetic applications). These will need to be sterilized using an appropriate method that does not adversely affect the properties of the material, and handled in a manner to ensure sterility.

4. Final Assembly

Electrical connections must be made to the active controlled release devices. This can be done after sealing and before the final sterilization of the device. For the study in Nature Biotechnology, 24:437-38 (April 2006), the microchip device was filled and sealed as a module, then attached to the electronics (physically and electrically). Since the reservoirs should be sealed hermetically, this process of attaching the filled and sealed module does not necessary need to be done in an aseptic environment, but should be done in one to reduce the bioburden for final sterilization.

After integration with the electronics, the device is given a final sterilization. The conditions of this sterilization must be compatible with each component of the whole device. For example, radiation generally would be unsuitable for a product incorporating a microchip with integrated electronic circuitry containing active components. However, if it is determined, for example, that active electronic components of the device can withstand sterilization by electron-beam, then such radiation sterilization would be preferable to other methods because of the ability of the radiation to penetrate through all of the materials of construction used in the product. This would permit one to fill, seal, and fully assemble a device without the cost and effort of performing these steps in an aseptic environment or using aseptic methods; one could therefore do a single e-beam sterilization of the final product, providing significant cost and timesavings.

Biosensor Devices

1. Sterilizing the Reservoir Device Body and Biosensor Substrate

The biosensors contained with the reservoir device body must be sterile as it will be in contact with the body once the reservoir containing the device is opened. Biosensors may be difficult to sterilize for implanted applications (von Woedtke, et al., “Sterilization of enzyme glucose sensors: problems and concepts”, Biosensors & Bioelectronics, 17:373-82 (2002)). They typically incorporate a biologically derived “recognition element” such as an enzyme, antibody or nucleic acid that confers specificity for the analyte of interest. An effective sterilization method disrupts the structure and function of these molecules. Most conventional, FDA-approved sterilization methods (i.e., dry heat, steam, ethylene oxide, radiation (gamma, e-beam), liquid chemical for single-use devices) affect the sensor response by damaging the “recognition element” and/or modifying the polymer membrane. Less traditional methods include light (high-intensity visible or UV), chlorine dioxide, vapor, gas plasma; these are often referred to as “cold” sterilization methods. The use of any of these methods will likely be accompanied by a loss in sensor performance which must be accounted for in the design of the sensor and sterilization process. Some sensor performance loss due to sterilization may be tolerable. The amount of performance that can be lost before the device becomes practically un-useable will be determined largely by the design of the sensor, the type and amount of recognition element present, and the other materials of the sensor.

As an alternative to the foregoing sterilization techniques, the biosensor may be prepared aseptically. Electrochemical biosensors are typically constructed by coating noble metal electrodes with sequentially deposited layers of biological and polymeric materials. The layers are prepared by solvent evaporation. The sensor substrate with electrodes can be sterilized with one of the traditional sterilization methods. Biological materials will generally be deposited from aqueous solutions and will need to be sterile filtered. Solutions of polymeric materials may be prepared in organic solvents which do not support bacterial growth and may not require sterile filtration (which could be difficult because these are often relatively viscous).

2. Sealing the Reservoirs

The sealing of the biosensor to the microchip must be carried out aseptically, unless one can use a penetrating form of radiation, such as electron-beam sterilization. Specialized equipment may be needed to join the previously sterilized components if a compression cold weld seal is used. Sterility assurance must be considered in the design and operation of this equipment.

3. Final Assembly

Generally, electrical connections will need to be made to both the microchip and the biosensor. The hermetic seal will prevent contamination of the reservoir and its contents, so an aseptic process is not needed. However, steps should be taken to minimize the bioburden for final sterilization. The choice of a terminal sterilization method must take into account the sensitivity of the various components to heat, radiation, etc.

Combination Biosensor/Drug Delivery Device

In applications where a monolithic device containing biosensors and pharmaceutical agents is made, the foregoing considerations for the preparation of sterile drug delivery and biosensor devices will apply. Generally, solid-state components such as the reservoir device body, reservoir caps, and sensor substrate can be sterilized by a number of traditional methods. Sterile filtration and dispensing in an aseptic environment typically will be required to maintain the activity of drug payloads and the sensor's biological “recognition elements.”

Publications cited herein are incorporated by reference. Modifications and variations of the methods and devices described herein will be obvious to those skilled in the art from the foregoing detailed description. Such modifications and variations are intended to come within the scope of the appended claims.

We claim:

1. A medical implant device having a mechanical wear detector comprising:
a prosthetic device body having at least one outer surface area;
at least one reservoir in the device body;
a wear indicator composition disposed in said at least one reservoir,
wherein mechanical wear of the at least one outer surface area of the device body in vivo causes release of at least part of the wear indicator composition.

2. The device of claim 1, wherein the prosthetic device body is a bone prosthesis or part thereof.

3. The device of claim 2, wherein the bone prosthesis is adapted for replacement of a hip, a knee, a shoulder, an elbow, or a vertebra.

4. The device of claim 1, wherein the device body and surface area in which the reservoirs are defined comprises a biocompatible material selected from metals, polymers, ceramics, and combinations thereof.

5. The device of claim 1, wherein the surface area comprises a polyethylene.

6. The device of claim 1, wherein the wear indicator composition comprises one or more matrix materials.

7. The device of claim 6, wherein the one or more matrix materials comprises a biodegradable, water-soluble, or water-swellable matrix material.

8. The device of claim 7, further a therapeutic or prophylactic agent in the matrix material degrades or dissolves in vivo to controllably release the therapeutic or prophylactic agent.

9. The device of claim 1, wherein the wear indicator composition is provided in two or more layers.

10. The device of claim 1, comprising a plurality of discretely spaced reservoirs.

11. The device of claim 10, wherein the reservoirs are micro-reservoirs.

12. The device of claim 10, wherein the reservoirs are formed in the device body by a microfabrication method.

13. An orthopedic implant device for controlled local release of a beneficial substance in vivo comprising:
a device body which comprises a release system which includes at least one beneficial substance,
wherein the beneficial substance is releasable from the device in vivo upon mechanical wear of at least one surface of the device body.

14. The device of claim 13, wherein the amount of beneficial substance released is proportional to the amount of mechanical wear experienced by the device body.

15. The implant device of claim 13, wherein the beneficial substance comprises a therapeutic or prophylactic agent.

16. The implant device of claim 15, wherein the therapeutic or prophylactic agent is a bisphosphonate.

17. The implant device of claim 13, wherein the beneficial substance comprises a biocompatible lubricating agent.

18. The implant device of claim 13, wherein the at least one beneficial substance is disposed in a plurality of discrete reservoirs located in the device body.

19. The implant device of claim 16, wherein the bisphosphonate is dispersed in a non-porous polymeric material which forms a wear surface on the device.

20. The implant device of claim 13, which is part of a knee implant, a hip implant, a bone resurfacing device, or an artificial vertebra.

21. The implant device of claim 13, further comprising:
at least one reservoir in the device body;
a wear indicator composition disposed in said at least one reservoir,
wherein mechanical wear of the at least one outer surface area of the device body in vivo causes release of at least part of the wear indicator composition.

22. A non-invasive method for detecting mechanical wear of a prosthetic device implanted in a human or other animal, the method comprising:
using a non-invasive imaging technique to image the prosthetic device which includes a wear indicating composition; and
detecting wear indicating composition that has been released from the prosthetic device.

23. The method of claim 22, wherein the imaging technique comprises magnetic resonance imaging, x-ray, ultrasound, positron emission tomography, or fluoroscopy.

24. The method of claim 22, wherein release of the wear indicating composition is detected by identifying the presence of at least a portion of the wear indicating composition at one or more positions remote from its original position in the prosthetic device.

25. The method of claim 22, wherein release of the wear indicating composition is detected by identifying the absence of at least a portion of the wear indicating composition from its original position in the prosthetic device.

26. The method of claim 22, wherein the prosthetic device includes wear indicating composition which is provided in each of a plurality of discrete reservoirs in the device.

27. The method of claim 26, wherein the reservoirs are microreservoirs.

28. The method of claim 22, which further comprises, before the step of using a non-invasive imaging technique, administering to the human or other animal a substance that interacts or binds with the wear indicating composition to enhance the detection of wear indicating composition that has been released from the prosthetic device.

29. The method of claim 28, wherein the non-invasive imaging technique comprises positron emission tomography and the substance comprises a radioactive agent.

30. A mechanical apparatus comprising:
a first structure having a wearable surface, which wears upon frictional engagement with a second structure during operation of the apparatus;
a plurality of discrete microreservoirs disposed in defined locations in the wearable surface; and
at least one wear indicating composition contained in the microreservoirs,
wherein upon a predetermined amount of wear of the wearable surface at least a portion of the at least one wear indicating composition is released from one or more of the microreservoirs.
